(19) United States
(12) Patent Application Publication
(10) Pub. No.: US 2026/0285558 A1
(43) Pub. Date: Sep. 24, 2026

(54) HYDRATION SENSING WATER BOTTLE WITH CONSUMPTION TRACKING AND MULTI-SENSOR CAP

(71) Applicant: Generosity Water, Inc., Montebello, CA (US)

(72) Inventors: Micah Cravalho, Los Angeles, CA (US); Brendan Lake, Venice, CA (US)

(73) Assignee: Generosity Water, Inc., Montebello, CA (US)

(21) Appl. No.: 19/674,919

(22) Filed: May 12, 2026

Related U.S. Application Data
(63) Continuation-in-part of application No. 19/061,482, filed on Feb. 24, 2025.
(60) Provisional application No. 63/558,392, filed on Feb. 27, 2024.
(60) Provisional application No. 64/012,499, filed on Mar. 21, 2026.

(51) Int. Cl. B65D 51/24; A45F 3/00; A45F 3/18; A61B 5/00; A61B 5/0531; G01F 23/292

(52) U.S. Cl. CPC B65D 51/245; A45F 3/18; A61B 5/0531; A61B 5/4875; A61B 5/6887; A61B 5/7275; G01F 23/292; A45F 2003/003

(57) ABSTRACT

A smart water bottle is operable to generate sensor data regarding the water in the bottle and transmit the data to at least one external server or device. The liquid level sensor is integrated into the cap and is a time of flight sensor operable to determine the distance of the surface of the water from the cap in order to determine volume. The smart water bottle further includes an exterior hydration sensor comprising LEDs, a light detecting module, and processor operable to determine hydration levels of a user. The smart water bottle further includes one or more pH sensors, and/or one or more total dissolved solids (TDS) sensors integrated into the cap. Additionally, a liquid intelligence platform, in communication with the smart water bottle, operable to calculate a net hydration score (NHS) and determine hydration levels based on data received from the smart water bottle.

### CROSS-REFERENCE TO RELATED APPLICATIONS

This application is related to and claims priority from the following US patents and patent applications: this application claims priority to and the benefit of U.S. Provisional Patent Application No. 64/012,499, filed Mar. 21, 2026, and is a continuation-in-part of U.S. patent application Ser. No. 19/061,482, filed Feb. 24, 2025, which claims priority to and the benefit of U.S. Provisional Patent Application No. 63/558,392, filed Feb. 27, 2024, each of which is incorporated herein by reference in its entirety.

### BACKGROUND OF THE INVENTION

### 1. Field of the Invention

The present invention relates to smart water bottles and a liquid intelligence system, and more specifically to water bottles with consumption volume, water quality, and hydration monitoring sensors in and/or on the bottle operable to communicate with a liquid intelligence system.

### 2. Description of the Prior Art

It is generally known in the prior art to provide smart water bottles and mobile applications for tracking water consumption over the course of a day.

Prior art patent documents include the following:
U.S. Pat. No. 11,013,353 for Wireless drink container for monitoring hydration by inventors Hambrock et al., filed Nov. 8, 2018, and issued May 25, 2021, discloses a wireless drink container able to monitor a person's hydration and prompt him or her to drink more if appropriate. The drink containers as described herein can monitor liquid levels and communicate with external devices about the liquid levels and rate of consumption. One or more sensors in the drink container monitor the liquid level within the container. A processor coupled to the sensor(s) estimates how much liquid has been removed from the container from changes in the liquid level and transmits a signal representing the change in liquid level to a smartphone or other external device. It also triggers an audio or visual indicator, such as an LED, that prompts the user to drink more based on the user's estimated liquid consumption and on the user's liquid consumption goals, which may be based on the user's physiology, activity level, and location.

U.S. Patent Pub. No. 2022/0218137 for Smart container with interactive, colored lights by inventors Iverson et al., filed Apr. 30, 2020, and published Jul. 14, 2022, discloses a smart container including a bottle or other container, a load cell, an accelerometer, a processor, and a colored light source. The container may be a liquid container, a pill container, or a food container. The colored light source may include one or more light-emitting diodes (LEDs) which can be programed to emit unique illumination patterns. The container may also include a speaker or motor to emit audio and vibrational notifications. The container provides a method of tracking consumption by a user of a substance held in a container.

U.S. Pat. No. 9,506,798 for Liquid consumption tracker by inventors Saltzgiver et al., filed Feb. 1, 2016 and issued Nov. 29, 2016, discloses a sensor operative to track changes in a liquid level of a hand-held liquid container, the sensor comprising: an ultrasonic liquid-level sensor configured to indicate a liquid level of a liquid in a hand-held liquid container, wherein the ultrasonic liquid-level sensor is capable of indicating the liquid level when the surface of the liquid is less than 20 centimeters away from the ultrasonic liquid-level sensor; a radio transmitter; and a processor configured to draw power from a portable power source, receive liquid-level indications from the ultrasonic liquid-level sensor, and cause the radio transmitter to transmit data indicative of changes in the liquid level.

U.S. Pat. No. 9,380,897 for Fluid consumption monitoring system by inventors Pfeiffer et al., filed Jan. 27, 2016 and issued Jul. 5, 2016, discloses an activity and volume sensing beverage container cap system for a beverage container including a cap that couples with the beverage container, a processor, a timer and at least one activity level sensor, such as an inclinometer, coupled with the processor. The activity level sensor detects container orientation.

When the container is tilted, the timer measures an amount of time in an orientation. Based on the current and previous orientation and the amount of time the container is in different orientations, the processor determines an amount of time of activity of the user, and a level of activity of the user, such as number of steps or moves per time. The inclinometer is also used to determine the amount of volume dispensed from the container when tilted over a threshold indicative of drinking. Thus one inclinometer may be utilized as both to determine activity level and volume of fluid dispensed.

U.S. Pat. No. 10,981,769 for Portable system for dispensing controlled quantities of additives into a beverage by inventors Lyons et al., filed Oct. 27, 2019, and issued Apr. 20, 2021, discloses a portable, self-contained beverage apparatus including a container assembly having a known storage capacity for storing a consumable liquid, and a dispensing assembly disposed within the container assembly that dispenses variable, non-zero quantities of additives into the consumable liquid. The dispensing assembly includes multiple apertures structured and arranged to retain vessels containing the additives to be dispensed into the consumable liquid. The beverage apparatus also includes a level sensor disposed within the container assembly that determines a consumable liquid level of the consumable liquid stored in the container assembly. In certain embodiments, one or more positive displacement pumping mechanisms are configured to pump additive liquid from additive containers into a beverage chamber.

WIPO Patent Pub. No. 2022/033666 for Smart bottle by inventors Villanueva Lopez et al., filed Aug. 11, 2020 and published Feb. 17, 2022, discloses a method for monitoring the use of a drinking bottle as well as a system for carrying out the method. According to the method a drinking bottle is provided which is designed to detect on basis of sensor data of at least one sensor whether the dispensing of liquid from the drinking bottle caused by drinking and to distinguish it from dispensing processes which do not represent drinking processes. In order to do so the evaluation system collects sensor data of the at least one sensor. The evaluation system evaluates this sensor data for detecting whether the dispensing has been caused by drinking and in case the dispensing has been caused by a drinking process stores the number of drinking processes and/or the volume of the liquid dispensing caused by drinking.

U.S. Pat. No. 11,185,179 for Liquid consumption monitoring device by inventor Zimbelman, filed Apr. 21, 2020 and issued Nov. 30, 2021, discloses a liquid consumption monitoring device for monitoring and encouraging consumption of a liquid including a cup and a lid, which is selectively engageable to the cup to close a top thereof. The user is positioned to tilt the cup to dispense a liquid from the cup, through an opening positioned in the lid, into a mouth of the user. A flow meter engaged to the cup, proximate to the opening, measures a volume of the liquid passing through the opening. An interface is engaged to at least one of the cup and the lid and is operationally engaged to the flow meter. The interface receives a signal from the flow meter when the liquid passes through the opening. The interface selectively actuates at least one of a speaker and a bulb, upon receipt of the signal from the flow meter, to provide a sensory reward to the user.

U.S. Patent Pub. No. 2022/0167129 for Electronically enabled drinking receptacles by inventor Yang, filed Nov. 24, 2021, and published May 26, 2022, discloses an electronically enabled drinking receptacle that has a liquid container, an electronic processor, an electronic drinking monitor, and a user-communicator. The drinking monitor can provide information to the electronic processor regarding an amount of liquid withdrawn from the liquid container over time and the electronic processor can be configured to actuate the user-communicator to communicate information to a user. The user-communicator can include one or more lights on the drinking receptacle, and the information communicated to the user can be configured to encourage the user to drink more liquid. The drinking receptacle can include an electronic communicator configured to communicate information between the liquid container and a separate electronic device, which can be remote from the drinking receptacle. The separate electronic device can be a mobile electronic device such as a wearable mobile electronic device.

U.S. Pat. No. 10,676,251 for Smart drink container by inventor Krafft, filed Oct. 27, 2016, and issued Jun. 9, 2020, discloses a container for storing a liquid for consumption. The container comprising temperature and volume sensors, processors, energy source and a communication device to transmit the recorded temperature and volume data to a remote human interface. The container may also provide for various notifications and alerts to the user if the data falls outside predetermined variable ranges.

U.S. Pat. No. 10,329,061 for System and methods for managing a container or its contents by inventors Dias et al., filed Nov. 6, 2014, and issued Jun. 25, 2019, discloses a retainer, a lid, and a sensor, where the sensor is configured to detect information about the retainer, the lid, or the contents in the retainer. The sensor also may be configured to communicate with an internal or external computer system, thereby facilitating showing the detected information as a representation via a display element. In certain embodiments, the system may include an action element such as an open/close lid opening assembly configured to permit automatically or manually opening or closing a drink aperture or another type of dispensing aperture.

It is also generally known in the prior art to provide a smart water bottle for tracking fluid intake metrics and to provide tests for water quality.

U.S. Pat. No. 10,433,666 for Liquid container systems and methods for monitoring user hydration by inventor Jovanov, filed Aug. 17, 2016, and issued Oct. 8, 2019, is directed to a smart beverage container that may be used to monitor user hydration. The container has at least one sensor for sensing an amount of liquid within the container. The liquid is monitored over time to determine an amount of liquid consumed by a user, and feedback is provided to the user indicating whether the user's liquid consumption is within a desired range according to a predefined profile of consumption. Such feedback may include information for indicating when the user is to consume additional liquid in order to remain in compliance with a desired liquid consumption regimen, as well as reminders to take additional liquid or warn the user if too much liquid had been consumed.

U.S. Patent Pub. No. 2024/0349919 for an Adapter ring for converting an ordinary water bottle into a smart water bottle by inventors Bono, et al., filed Apr. 19, 2024, and published Oct. 24, 2024, is directed to an adapter ring securable to a new or pre-existing water bottle that provides monitoring of the liquid inside of the water bottle and wireless communication to an external electronic device, which may include an algorithm to help the user maintain proper hydration. A bottom portion of the adapter ring includes external or internal helical threads, or the like, adapted to fit onto a threaded portion of the water bottle or container. A top portion of the adapter ring also includes threads, or the like, for connection to the original water bottle top. The adapter ring includes a sealed chamber having a wall separating the liquid flow path from the electronics so that the liquid flow path provides a fluid communication between the top portion and bottom portion of the adapter ring to allow for consumption of the liquid in the container.

U.S. Pat. No. 12,082,729 for a Smart container with interactive, colored lights by inventors Iverson, et al., filed Oct. 29, 2021, and issued Sep. 10, 2024, is directed to a smart container that includes a bottle or other container, a load cell, an accelerometer, a processor, and a colored light source. The container may be a liquid container, a pill container, or a food container. The colored light source may include one or more light-emitting diodes (LEDs) which can be programed to emit unique illumination patterns. The container may also include a speaker or motor to emit audio and vibrational notifications. The container provides a method of tracking consumption by a user of a substance held in a container.

U.S. Pat. No. 10,750,842 for a Bottle by inventors Sengupta, et al., filed Oct. 12, 2018, and issued Aug. 25, 2020, is directed to a smart and intelligent water bottle that can inform its user of his hydration needs by tracking water consumed by the user and also functions as a safety device by enabling user to transmit a SoS and GPS co-ordinates to predefined list of friends, family or official institutions. The bottle, while tracking water consumption, can differentiate between actual consumption and intended or unintended discharge of its contents. The bottle incorporates a panic button configured to recognize a genuine pressing of the panic button based on natural increase in heart rate of the user in case of emergencies. The disclosed bottle incorporates a capacitive sensor and a tilt sensor for measurement of water present in the bottle is done. In an aspect, the capacitive sensor is configured centrally within a hollow space in the bottle to prevent its contact with water.

U.S. Patent Pub. No. 2024/0389940 for a Hydration Monitoring Device and System by inventors Diaz, et al., filed May 24, 2024, and published Nov. 28, 2024, is directed to a hydration monitoring device and system. The hydration monitoring device includes housing having a base with a receptacle for holding a water bottle, a weight sensor to detect changes in the water bottle's weight, and a transceiver for connectivity with an application on an electronic device. A display is disposed on an exterior of the housing and a speaker for delivering visual and audible notifications. The display is a touchscreen to provide a user interface for allowing the user to customize notifications and settings. The weight sensor tracks water consumption, and the system calculates a desired hydration rate based on user data and activity levels. The device and system provide each provide visual and auditory prompts to the user when water consumption falls below the target rate, ensuring adequate hydration. This system facilitates real-time monitoring and encourages consistent water intake, enhancing user health and well-being.

U.S. Pat. No. 12,256,857 for an Advanced intelligent fluid bottle apparatus, method, and system by inventor Murry, filed Sep. 5, 2022, and issued Mar. 25, 2025, is directed to the first seriously smart water bottle. The bottle is designed to provide an easier, more accurate approach to tracking and logging one's water/fluid intake with minimal effort from the user. Mass utilization of this environmental-friendly hydration tool will also serve as a catalyst for the promotion of environmental consciousness and add value by helping push the conversation forward about global environmental protection. Variations of this mechanism will grant anyone, with a smart device, the ability to monitor the amount of water/fluid consumption, not only for themselves, but also for anyone else they care for, from infants to senior citizens. The device will operate in conjunction with its namesakes' software application. Regardless of gender, age, height, weight, health condition, or activity level this tool will undoubtedly aid the user in leading a healthier, more productive lifestyle and in some cases, be essential to the preservation of life.

U.S. Pat. No. 12,405,184 for a Water meter and leak detection system by inventor Klicpera, filed Nov. 22, 2023, and issued Sep. 2, 2025, is directed to a water meter collection node (i.e., water meter or base station) with a water flow rate sensor(s), water valve control mechanism (water shut-off/on), and leak detection capability. The system uses wireless transceiver technology to communicate with a private or corporate network, or to communicate via an internet connection. The system transfers encrypted water data and information via application programming interface to remote computers and/or server, enabling access of the water data and information by remote electronic communication devices. The water meter collection node is configured to communicate with an authenticated communication hub that functions to extend the range of wireless transmissions of the collection node. The communication hub can by wire or wirelessly communicate with an internet router and transfers water data and information to an internet connection. The collection node(s) and/or communication hub(s) can wirelessly communicate with a private or commercial network system, and to a remote computer/server and/or a cloud service. One or more communication hubs can function as repeater apparatuses and/or can perform complex operations such as using mesh technology, or point-to-point or peer-to-peer connections.

U.S. Pat. No. 12,080,147 for Determining alternative outcome or event based on aggregated data by inventors Vadwa, et al., filed Dec. 18, 2020, and issued Sep. 3, 2024, is directed to a data collection device and method for determining an alternative event condition. The method includes receiving a first data communication including a first data value from a first sensor and determining a first event condition based on the first data value being beyond a predetermined threshold. The method further includes receiving additional data communications including additional data values from one or more second sensors, aggregating the first data value and the additional data values, and determining that the determination of the first event condition is incorrect based on the aggregated data values and a known relationship between the first sensor and the one or more second sensors. The method additionally includes determining a second event condition based on the first data value and the additional data values being beyond the predetermined threshold. An action may then be performed in response to the determination of the second event condition.

### SUMMARY OF THE INVENTION

The present invention relates to smart water bottles and a liquid intelligence platform, and more specifically to water bottles with consumption volume, water quality, and hydration monitoring sensors in the bottle operable to communicate with a liquid intelligence system.

It is an object of this invention to provide a smart water bottle having integrated sensors for tracking consumption, hydration, and other liquid qualities such as pH, and water intelligence platform to provide real-time hydration and other data.

In one embodiment, the present invention includes a water bottle, including a water bottle body including a consumption verification module, a water bottle cap configured to attach to a top of the water bottle body, a sensor cradle extending downwardly from a bottom surface of the water bottle cap, wherein the sensor cradle includes a plurality of prongs, a plurality of total dissolved solids (TDS) sensors embedded in or attached to tips of the plurality of prongs, and a time of flight sensor embedded in the sensor cradle and surrounded by the plurality of prongs, wherein the consumption verification module comprises an accelerometer and a gyroscope, wherein the consumption verification module is configured to provide movement metrics and indicate whether or not the water bottle is resting or moving, and wherein the water bottle is configured to perform intake event verification to verify when fluid intake occurs and the amount of fluid ingested based on the consumption verification module, the plurality of TDS sensors, and the time of flight sensor.

In another embodiment, the present invention includes a water bottle cap, including a consumption verification module comprises an accelerometer and a gyroscope, wherein the consumption verification module is configured to provide movement metrics and indicate whether or not the water bottle is resting or moving, a sensor cradle extending downwardly from a bottom surface of the water bottle cap, wherein the sensor cradle includes a plurality of prongs, a plurality of total dissolved solids (TDS) sensors embedded in or attached to tips of the plurality of prongs, and a time of flight sensor embedded in the sensor cradle and surrounded by the plurality of prongs, wherein the water bottle cap is configured to attach to a top of a water bottle body, and wherein the water bottle is configured to perform intake event verification to verify when fluid intake occurs and the amount of fluid ingested based on the consumption verification module, the plurality of TDS sensors, and the time of flight sensor.

In yet another embodiment, the present invention includes A water bottle, including a water bottle body including a consumption verification module, a water bottle cap configured to attach to a top of the water bottle body, a water bottle base configured to attach to a bottom of the water bottle body, one or more total dissolved solids (TDS) sensors, one or more time of flight sensors, one or more pH sensors, and/or one or more temperature sensors included within the water bottle cap, one or more intra-device antennas positioned in the water bottle cap, one or more external network antennas positioned in the water bottle base, wherein the one or more intra-device antennas are configured to transmit sensor data generated by the one or more total dissolved solids (TDS) sensors, the one or more time of flight sensors, the one or more pH sensors, and/or the one or more temperature sensors to the water bottle base, wherein the consumption verification module comprises an accelerometer and a gyroscope, wherein the consumption verification module is configured to provide movement metrics and indicate whether or not the water bottle is resting or moving, wherein the one or more sensors momentarily stop reading upon the movement metrics indicating a moving water bottle, and wherein the one or more sensors resume reading upon the movement metrics indicating a resting water bottle, wherein the water bottle is configured to perform intake event verification to verify when fluid intake occurs and the amount of fluid ingested based on the consumption verification module and the sensor data, and wherein the one or more external network antennas are configured to transmit the sensor data to one or more external devices and/or one or more external servers.

These and other aspects of the present invention will become apparent to those skilled in the art after a reading of the following description of the preferred embodiment when considered with the drawings, as they support the claimed invention.

### BRIEF DESCRIPTION OF THE DRAWINGS

FIG. 1A illustrates a perspective view of a bottle according to one embodiment of the present invention.

FIG. 1B illustrates a side view of a bottle according to one embodiment of the present invention.

FIG. 2A illustrates a bottom perspective view of an intelligent bottle cap according to one embodiment of the present invention.

FIG. 2B illustrates a side sectional view of an intelligent bottle cap according to one embodiment of the present invention.

FIG. 2C illustrates a bottom orthogonal view of an intelligent bottle cap according to one embodiment of the present invention.

FIG. 2D illustrates a side orthogonal view of an intelligent bottle cap according to one embodiment of the present invention.

FIG. 3 illustrates a side sectional view of an intelligent bottle cap connected to a water bottle according to one embodiment of the present invention.

FIG. 4 illustrates a perspective sectional view of a basic bottle cap according to one embodiment of the present invention.

FIG. 5 illustrates a perspective sectional view of an intelligent bottle cap according to one embodiment of the present invention.

FIG. 6 illustrates a perspective view of a bottle cap and a corresponding charging system according to one embodiment of the present invention.

FIG. 7 illustrates a conceptual diagram of the electronic components of a bottle cap according to one embodiment of the present invention.

FIG. 8A illustrates a side view of a bottle including a hydration sensor according to one embodiment of the present invention.

FIG. 8B illustrates a side view of a bottle including a handle and hydration sensor according to one embodiment of the present invention.

FIG. 8C illustrates a side view of a bottle including a bottle cap with a hydration sensor according to one embodiment of the present invention.

FIG. 9 illustrates a conceptual diagram of an intelligent water bottle connected to a network according to one embodiment of the present invention.

FIG. 10 illustrates a perspective view of an intelligent bottle cap according to one embodiment of the present invention.

FIG. 11 illustrates a diagram including three layers of the liquid intelligence platform, according to one embodiment of the present invention.

FIG. 12 illustrates a block diagram of communication between the liquid intelligence platform and the intelligent water bottle, according to one embodiment of the present invention.

FIG. 13 illustrates a graphical user interface (GUI) for a dashboard of the liquid intelligence platform, according to one embodiment of the present invention.

FIG. 14 illustrates a graphical user interface (GUI) for the liquid test section of the liquid intelligence platform, according to one embodiment of the present invention.

FIG. 15 illustrates a graphical user interface (GUI) for a quick response (QR) scanner of the liquid intelligence platform, according to one embodiment of the present invention.

FIG. 16A illustrates a graphical user interface (GUI) for a hydration station navigation of the liquid intelligence platform, according to one embodiment of the present invention.

FIG. 16B illustrates a graphical user interface (GUI) for the hydration station navigation of the liquid intelligence platform, according to one embodiment of the present invention.

FIG. 16C illustrates a graphical user interface (GUI) for the hydration station navigation of the liquid intelligence platform, according to one embodiment of the present invention.

FIG. 17 illustrates a graphical user interface (GUI) for the contaminants page of the liquid intelligence platform, according to one embodiment of the present invention.

FIG. 18 illustrates a graphical user interface (GUI) for a starting page of the liquid intelligence platform, according to one embodiment of the present invention.

FIG. 19 is a schematic diagram of a system of the present invention.

### DETAILED DESCRIPTION

The present invention is generally directed to smart water bottles and a liquid intelligence system, and more specifically to water bottles with consumption volume, pH, and hydration monitoring sensors in and/or on the bottle operable to communicate with a liquid intelligence system.

In one embodiment, the present invention includes a water bottle, including a water bottle body including a consumption verification module, a water bottle cap configured to attach to a top of the water bottle body, a sensor cradle extending downwardly from a bottom surface of the water bottle cap, wherein the sensor cradle includes a plurality of prongs, a plurality of total dissolved solids (TDS) sensors embedded in or attached to tips of the plurality of prongs, and a time of flight sensor embedded in the sensor cradle and surrounded by the plurality of prongs, wherein the consumption verification module comprises an accelerometer and a gyroscope, wherein the consumption verification module is configured to provide movement metrics and indicate whether or not the water bottle is resting or moving, and wherein the water bottle is configured to perform intake event verification to verify when fluid intake occurs and the amount of fluid ingested based on the consumption verification module, the plurality of TDS sensors, and the time of flight sensor.

In another embodiment, the present invention includes a water bottle cap, including a consumption verification module comprises an accelerometer and a gyroscope, wherein the consumption verification module is configured to provide movement metrics and indicate whether or not the water bottle is resting or moving, a sensor cradle extending downwardly from a bottom surface of the water bottle cap, wherein the sensor cradle includes a plurality of prongs, a plurality of total dissolved solids (TDS) sensors embedded in or attached to tips of the plurality of prongs, and a time of flight sensor embedded in the sensor cradle and surrounded by the plurality of prongs, wherein the water bottle cap is configured to attach to a top of a water bottle body, and wherein the water bottle is configured to perform intake event verification to verify when fluid intake occurs and the amount of fluid ingested based on the consumption verification module, the plurality of TDS sensors, and the time of flight sensor.

In yet another embodiment, the present invention includes A water bottle, including a water bottle body including a consumption verification module, a water bottle cap configured to attach to a top of the water bottle body, a water bottle base configured to attach to a bottom of the water bottle body, one or more total dissolved solids (TDS) sensors, one or more time of flight sensors, one or more pH sensors, and/or one or more temperature sensors included within the water bottle cap, one or more intra-device antennas positioned in the water bottle cap, one or more external network antennas positioned in the water bottle base, wherein the one or more intra-device antennas are configured to transmit sensor data generated by the one or more total dissolved solids (TDS) sensors, the one or more time of flight sensors, the one or more pH sensors, and/or the one or more temperature sensors to the water bottle base, wherein the consumption verification module comprises an accelerometer and a gyroscope, wherein the consumption verification module is configured to provide movement metrics and indicate whether or not the water bottle is resting or moving, wherein the one or more sensors momentarily stop reading upon the movement metrics indicating a moving water bottle, and wherein the one or more sensors resume reading upon the movement metrics indicating a resting water bottle, wherein the water bottle is configured to perform intake event verification to verify when fluid intake occurs and the amount of fluid ingested based on the consumption verification module and the sensor data, and wherein the one or more external network antennas are configured to transmit the sensor data to one or more external devices and/or one or more external servers.

The utilization of data tracking in managing everyday life has had benefits particularly in the health industry. The integration of either sensors or self-input data into web or mobile applications has allowed for more comprehensive and accurate diet tracking, fitness routines, and sleep quality tracking in ways previously not possible. One area of health that holds particular importance is hydration, not only for those regularly engaged in strenuous workouts, but even for office workers who often forget or otherwise neglect to consume enough water to maintain a healthy lifestyle.

One approach to hydration management is to use mobile applications for manual logging and for user notifications. For example, apps such as WATERLLAMA allow a user to log types of beverages being drunk and sends notifications based on physical qualities of the user, such as sex, weight, daily activity, and weather to determine how often the user needs to drink. Much of this functionality, namely manual tracking and using health and personal data to determine a routine are common among a variety of apps including HYDRO COACH, WATERMINDER, and MY WATER, among other applications. However, apps such as these are reliant on user participation and engagement to generate data. Even missing a single day damages the usefulness of a longer period of data, especially if missed days tend to be associated with days with specific compliance levels, such as days that tend to be missed being low compliance days, which skews the data. While for some users, missing data does not represent a serious issue, for those with known health issues or with strenuous exercise requirements, such skewed data is capable of having significant health implications.

A system integrating actual sensor data corresponding to water consumption from at least one source (e.g., a water bottle) is important for measuring actual compliance with a hydration routine, even on days when an individual does not have the time or otherwise does not remember to log water consumption. Some systems have attempted to integrate sensors and sensor data transmission into water bottles, such as U.S. Pat. No. 11,013,353. The '353 patent includes a liquid level sensor disposed within the cavity of the bottle and an accelerometer to determine when the container tips. However, the '353 patent extolls the benefits of the use of such sensors as capacitive sensor or Hall effect sensors for using less power than other sensors for the liquid level sensor.

Capacitive fluid measurements and Hall effect sensors, however, require specific orientations of the fluid in the container and often fail to detect liquid levels when the levels are low in the container.

Furthermore, rather than being integrated into the cap, these sensors require a long extension to extend into the chamber in order to operate, which both limits the volume of water contained and requires an overly bulky and more easily damageable mechanism.

While some patents such as U.S. Pat. No. 9,506,798 mention use of an ultrasonic liquid level sensor for use in a water bottle cap, but only for the purpose of detecting liquid less than 20 centimeters away from the sensor, these systems, as well as those describing other types of liquid level sensor, fail to describe a reasonable method to charge or otherwise power the sensor devices.

Instead, these systems either assume low power draw after which the caps or entire bottles are to be discarded, or describe systems with replaceable batteries. Furthermore, these systems do not provide any method to test the pH or total dissolved solids (TDS) of the water within the bottle, and therefore fail to monitor important quantities of the water.

The present invention further provides useful information for hydration in particular, as it accounts for both the TDS and quantity of water consumed. Because TDS content impacts the actual hydration provided by the water, providing information regarding both the TDS and the quantity of water consumed provides more detailed information regarding the actual hydration of the user than is provided by the prior art.

Smart water bottles commonly include mechanisms to determine the remaining amount of water in the bottle based on the water height or the weight of the water relative to the water bottle. In the prior art, smart water bottles are inoperable to calculate the water quality of the water contained in the bottle. Traditional smart water bottles generally measure the amount of water removed from the smart water bottle in combination with a recommended water consumption schedule to determine a hydration level of a user. Problematically, these traditional mechanisms determine hydration levels based solely on water consumption but fail to account for other significant hydration level metrics, such as water quality, electrolyte absorption, fluid intake from food, aggregated local municipality source water profiles, and fluid loss.

Generally, existing systems use an external application on a computing device for water intake scheduling to ensure hydration or to avoid dehydration. The external application adjusts the frequency of scheduled water intake based on individualized hydration goals. Water intake scheduling simply provides a timer system to indicate designated hydration periods and does not provide accurate hydration measurements.

Smart water bottles that determine a hydration level based on a water intake schedule and remaining water neglect other necessary metrics for hydration, including water loss, water quality, and other parameters. Therefore, there is an unmet need for an intelligent system that determines hydration levels based on an analysis of detailed user metrics. There is also a need for optimizing water intake scheduling, mechanisms for detailing water quality measurement, and optimizing high-level intelligence systems to track external user metrics and accurately compute a hydration level. Furthermore, there is a need for an intelligent system that is operable to analyze water attributes based on data received from a sensor in real-time or near real-time and historical data from municipalities and drinking water sources to determine water quality and provide recommendations for treatment.

None of the prior art discloses an intelligent water bottle including an intelligent bottle cap with nested sensors for determining water quality parameters. Furthermore, none of the prior art discloses a liquid intelligence system in communication with an intelligent water bottle, a smart device, and/or one or more sensors in the intelligent water bottle a water supply system or end points of a water supply system, such as a water fountain, tap, under the sink unit, or whole house filtration unit, that provides a hydration score based on fluid intake, water loss, and hydration goals optimized based on holistic metrics including aggregated metrics from a municipality. The system of the present invention provides for determination and visualization of the relationship between treatment-system performance, water composition, absorption efficiency, and resulting hydration state. The platform offers insights regarding absorption efficiency based on absorption efficiency models and hydration-state intelligence, which are more relevant health metrics than just intake concentration models and consumption logging. Additionally, none of the prior art discloses a multi-layer liquid intelligence platform operable to aggregate data from smart devices and municipalities to provide reports regarding water quality that provide for improving water quality parameters and improved hydration score computations.

Referring now to the drawings in general, the illustrations are for the purpose of describing one or more preferred embodiments of the invention and are not intended to limit the invention thereto.

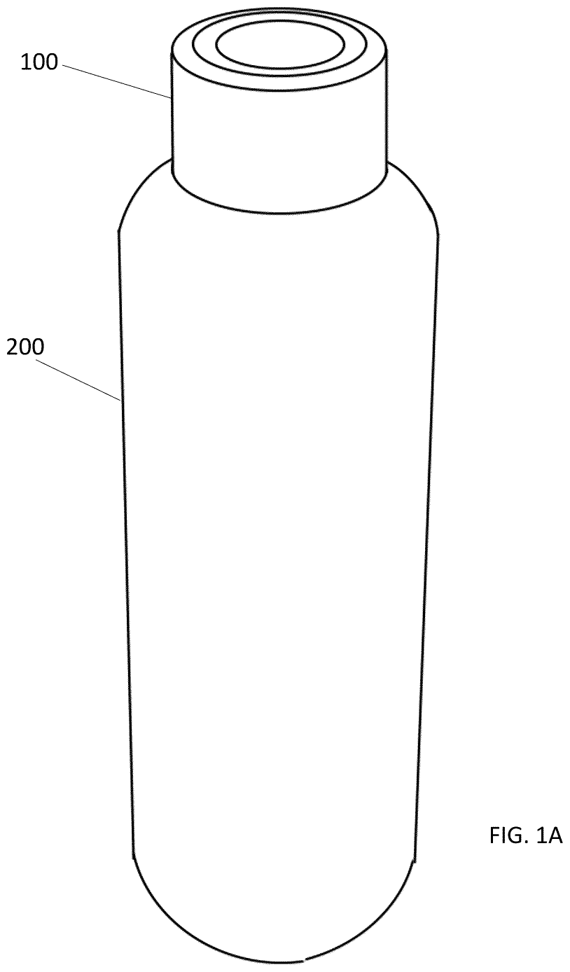

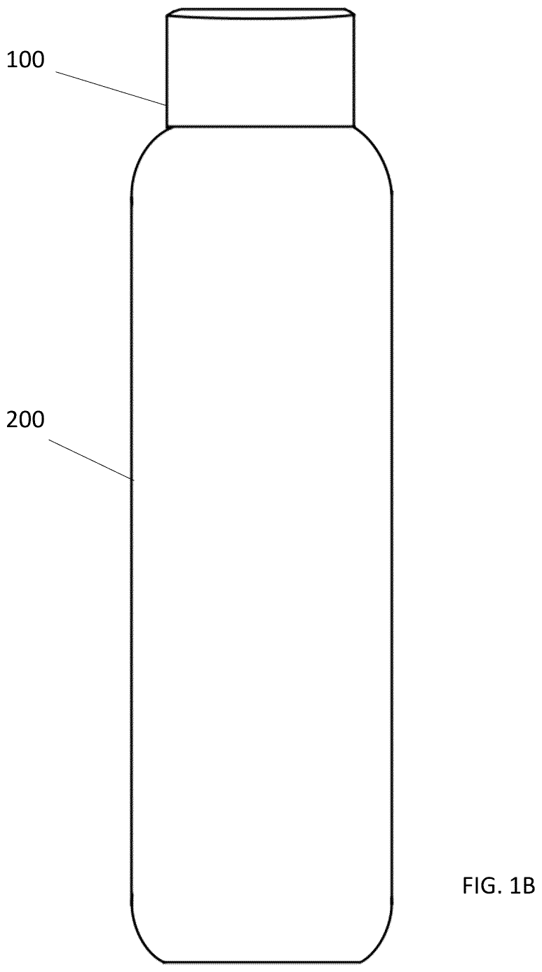

FIGS. 1A-1B illustrate a bottle according to one embodiment of the present invention. A water bottle includes a vessel 200 capped by a bottle cap 100 at a top end. The bottle of the present invention may be referred to as water bottle, intelligent water bottle, and/or smart water bottle herein, as these terms are interchangeable for every embodiment of the present invention. In one embodiment, the vessel 200 is substantially cylindrical. In one embodiment, the bottle cap 100 is, similarly, substantially cylindrical. However, one of ordinary skill in the art will understand that other shapes for the water bottle (e.g., rectangular prismatic, etc.) are also contemplated and compatible with the present invention.

In one embodiment, the vessel 200 is formed from at least one metal material, including, but not limited to, stainless steel, aluminum, and/or at least one metal alloy. In one embodiment, the vessel 200 is formed from at least one composite material, including, but not limited to, carbon fiber and fiberglass. In one embodiment, the vessel 200 is formed from at least one hard polymer material, including Tritan, polypropylene (PP), polyethylene terephthalate (PET), polycarbonate (PC), high-density polyethylene (HDPE), and/or one or more other hard polymer materials, wherein the at least one hard polymer material is food-grade material. In another embodiment, an intelligent bottle cap, as disclosed in further embodiments, is formed from the at least one hard polymer material. In one embodiment, the vessel 200 is formed from glass. In one embodiment, the inner surface and/or the out surface of the vessel 200 is coated with at least one powder coating and/or at least one silicone coating. In one embodiment, the vessel 200 includes a double wall design, but one of ordinary skill in the art will understand that other designs, including, but not limited to, single wall and triple wall configurations are also contemplated herein. In one embodiment, the materials and construction of the vessel 200 is configured to provide at least a 24-hour rating for cold fluids and/or at least a 12-hour rating for hot fluids. In one embodiment, the bottle is configured to hold approximately 1 liter, but one of ordinary skill in the art will understand that the bottle cap 100 of the present invention is operable to be affixed to different water bottles of various volumes. One of ordinary skill in the art will understand that the exterior of the bottle is able to include one or more shades of white, brown, red, pink, blue, black, and/or one or more shades of any other color, as the color is not intended to be limiting.

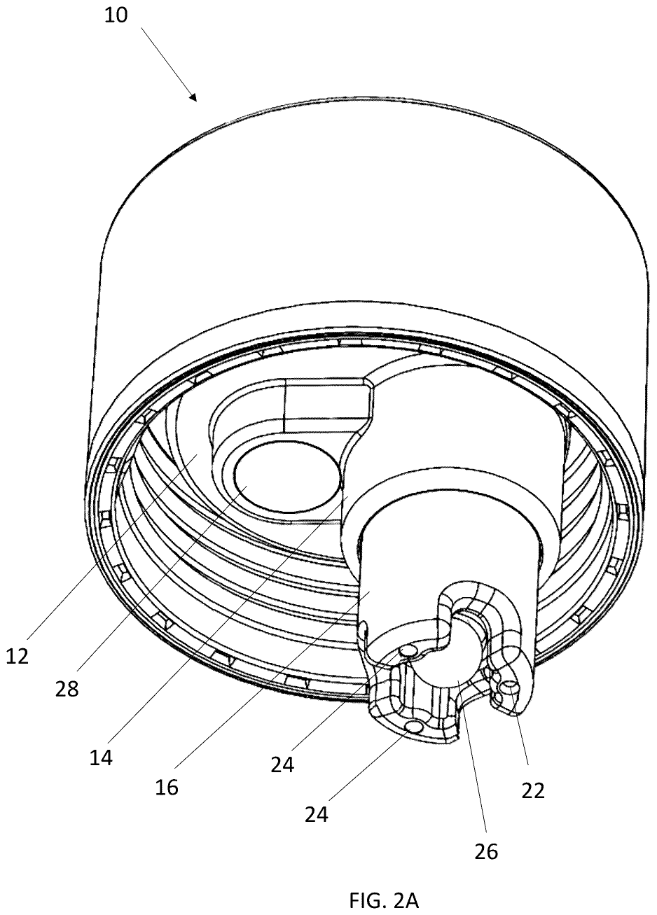

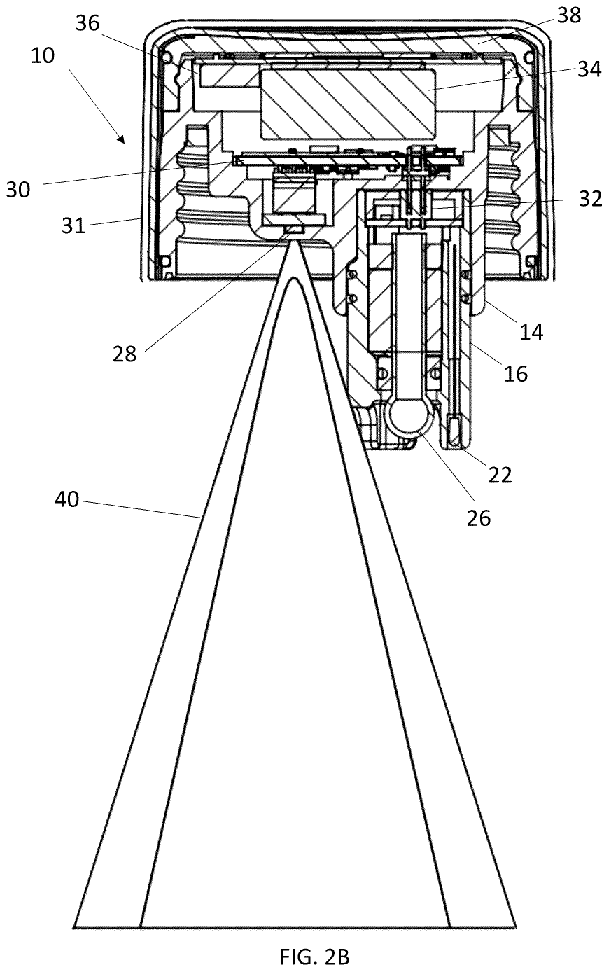

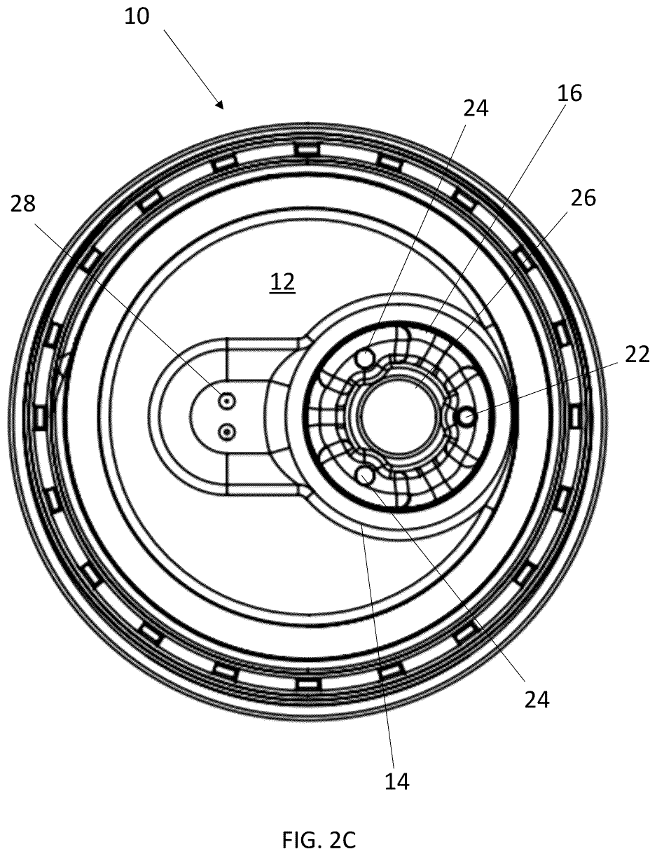

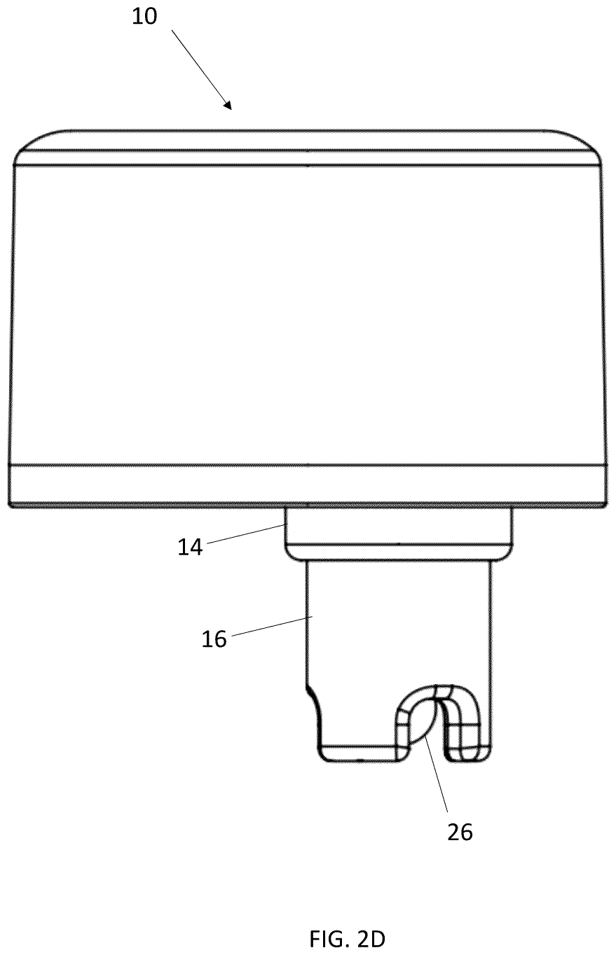

FIG. 2A illustrates a bottom perspective view of an intelligent bottle cap according to one embodiment of the present invention. The intelligent bottle cap 10 shown in FIG. 2A includes a bottom recession, including a plurality of threads lining an interior wall of the bottom recession, such that the bottle cap 10 is able to matingly fit with exterior threads on a top exterior surface of a water bottle. Alternatively, one of ordinary skill in the art will understand that the intelligent bottle cap 10 is instead able to include threads lining an exterior surface of the bottle cap 10 around the bottom recession, such that bottom of the bottle cap 10 is able to fit within an opening at the top of the water bottle and such that the threads lining the exterior surface of the bottle cap 10 matingly pair with threads lining an interior surface of the opening of the water bottle.

In an alternative embodiment, an intelligent lid is operable to matingly pair with any vessel (e.g., cup, jar, etc.), wherein the intelligent lid is operable to comprise all components and execute every function of the intelligent bottle cap 10 as described herein.

In a preferred embodiment, a sensor cradle 14 extends downwardly from a top surface 12 of a recession in the bottom of the bottle cap 10. In one embodiment, the sensor cradle 14 is configured to retain at least one time of flight sensor 28. In one embodiment, the at least one time of flight sensor 28 is aimed at and operable to detect time of flight (and correspondingly distances) along a longitudinal axis of the bottle cap 10 (i.e., toward the bottle to which the cap 10 is affixed).

Therefore, the time of flight sensor 28 is operable to detect the distance between the bottle cap 10 and the surface of a fluid filling the bottle, thereby allowing the water level and volume of water within the bottle to be determined. In one embodiment, the at least one time of flight sensor comprises a single-photon avalanche diode (SPAD) based laser operable to detect time of flight.

In one embodiment, the sensor cradle 14 also retains or is integrally formed with an extension 16 extending downwardly from the top surface 12 of the recession in the bottom of the bottle cap 10. A plurality of sensors are positioned at the bottom of the extension 16, and is configured to be of sufficient length such that extends beyond the bottom of the bottle cap 10. This allows the extension to be proximate to or positioned within the water or other liquid held in a bottle to which the bottle cap 10 is affixed. In one embodiment, one or more pH sensors 26 extends from approximately a center bottom section of the extension 16. In one embodiment, one or more TDS sensors 24 are attached to the bottom of the extension 16. In a preferred embodiment, two TDS sensors 24 are attached to the bottom of the extension 16. In one embodiment, one or more temperature sensors 22 are attached to the bottom of the extension 16. In one embodiment, as shown in FIG. 2A, three smaller prongs extend from an outer perimeter of a bottom end of the extension 16, two of which holds TDS sensors 24 and one of which holds a temperature sensor 22, with the three smaller prongs surrounding a central pH sensor 26. This three-prong configuration provides for improved protection of the central pH sensor 26 from, among other scenarios, bumping the extension 16 against the bottle or objects, and providing protection from debris coating the central pH sensor 26. The combination of both pH sensors and temperature sensors in the present invention is important for accurately determining the pH of the water, as the pH detected by the sensors is dependent on the temperature of the water. Therefore, in one embodiment, the bottle cap 10 includes a processor operable to receive data from both the central pH sensor 26 and the temperature sensor 22 and determine the actual pH of the water or other fluid in the bottle.

In one example embodiment, the one or more temperature sensors 22 are operable to measure from a range of −5 to 215 degrees Fahrenheit (approximately 20 to 100 degrees Celsius). In another embodiment, the one or more temperature sensors 22 are operable to measure from any range of temperatures. In one embodiment, the one or more temperature sensors 22 are operable to detect a change in temperature per 0.1 degrees. In another embodiment, the one or more temperature sensors 22 are operable to detect a change in temperature per 0.01 degrees. In one embodiment, the one or more temperature sensors 22 further include 316 pins. In one embodiment, the one or more temperature sensors 22 are operable to measure temperature with a 2% margin of error. In another embodiment, the one or more temperature sensors 22 are operable to measure temperature with less than a 2% margin of error.

FIG. 2B illustrates a side sectional view of an intelligent bottle cap according to one embodiment of the present invention. In an alternative embodiment, the intelligent bottle cap further includes one or more pH sensors 26. As shown in FIG. 2B, the one or more pH sensors 26 are embedded in approximately the center of the extension 16, with a bulb of the one or more pH sensors 26 exposed at the bottom of the extension 16 in order to allow exposure to the water. Similarly, the temperature sensor 22 extends through an edge of the extension 16 such that it is exposed at the bottom.

The one or more pH sensors 26 and the other sensors within the extension 16 are connected to a central control board 30 of the bottle cap 10 via a plug 32 extending into an opening of the sensor cradle 14. In one embodiment, the extension 16 is easily detachable from the sensor cradle 14 via plugging and unplugging the extension 16 into the sensor cradle 14. This allows the extension 16, and the associated sensors within the extension 16, to be easily replaced or cleaned due to debris or other potential damage to the sensors in the extension 16.

The offset between the time of flight sensor 28 and the extension 16 is important. The cone of vision 40 of the time of flight sensor 28 is shown in FIG. 2B, with the offset of the extension 16 allowing the full cone of vision 40, or substantially the full cone of vision 40, to detect the distance to the water in the bottle. In one embodiment, the time of flight sensor 28 includes at least one lens configured to limit the cone of vision 40 of the time of flight sensor 28 to effectively focus the time of flight sensor 28 downward toward the surface of the water. In a preferred embodiment, the intelligent bottle cap 10 includes two time of flight sensors 28 directly adjacent to each other and each offset from the extension 16. As seen in FIG. 2B, the time of flight sensor 28 is also connected to the central control board 30.

In one embodiment, the bottle cap 10 includes a battery 34 positioned above the central control board 30. This is particularly important in embodiments where the bottle cap 10 is configured for wireless charging through a top end of the cap 10, as the battery 34 is positioned proximate to the top of the cap 10. In one embodiment, a speaker 36 (e.g., a piezo speaker) is positioned within the cap 10 proximate to the battery 34, near the top of the cap 10, as shown in FIG. 2B. In one embodiment, the speaker 36 is able to provide auditory indications of a state of the cap 10 (e.g., battery state, connectivity state, etc.) or of the water within the bottle (e.g., a quality of water, a volume of water, etc.). In one embodiment, the battery 34 is a 450 milli-ampere hour (mAh) lithium polymer (LiPo) battery or any other capacity of battery power and material, wherein the battery 34 is operable to supply power for at least 2.5 hours. In another embodiment, the battery 34 has a standby time of at least 300 days. In another embodiment, the battery 34 has a standby time of any number of days, months, and/or years.

In one embodiment, the bottle cap 10 further includes a BLUETOOTH low energy (BLE) module operable to transmit and receive BLUETOOTH signals. In a preferred embodiment, the BLE module comprises an nRF 52832 module. In one embodiment, the BLE module is BLUETOOTH 5.0 compatible. In another embodiment, the BLE module is compatible with any version of BLUETOOTH.

In one embodiment, an upper shell 38 of the bottle cap 10 is formed from a non-metal material and/or a material that allows for the transmission of BLUETOOTH signals (or other wireless network signals) into and out of the bottle cap 10. This is important, as it allows for the transmission of commands into the cap 10 and/or the transmission of information out of the cap 10. In one embodiment, the upper shell 38 is substantially translucent or transparent, allowing light emitting diodes (LEDs) within the cap 10 to shine through the top of the cap 10. These LEDs are able to provide indications of a state of the cap 10 (e.g., battery state, connectivity state, etc.) and/or of the water within the bottle to which the cap 10 is attached (e.g., a quality of water, a volume of water, etc.).

Additional views of the cap 10 are shown in FIGS. 2C and 2D.

In one embodiment, the cap 10 is overmolded with silicone 31 or another similar material. The overmolded silicone 31 provides an additional dust seal between the lid and the bottle when the lid is screwed on. In another embodiment, the base of the vessel further includes the overmolded silicone.

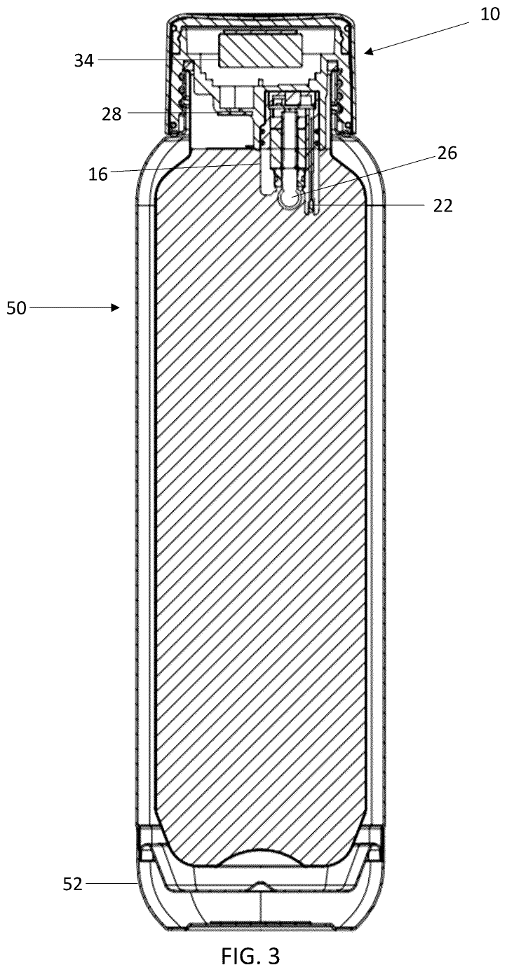

FIG. 3 illustrates a side sectional view of an intelligent bottle cap 10 connected to a water bottle 50 according to one embodiment of the present invention. As shown in FIG. 3, when the intelligent bottle cap 10 is attached to a bottle 50 and the bottle 50 is substantially full of water or other liquid, both the pH sensor 26 and the temperature sensor 22 extend into the water such that quality data is able to be generated. Additionally, though it is not shown in FIG. 3, the TDS sensor also ideally extends into the water or other liquid when in this state. In another embodiment, the TDS sensor is operable to detect a range from 0 to 3,000 parts per million (ppm) of TDS. In another embodiment, the TDS sensor includes 316 stainless pins on the cradle prongs. In another embodiment, the TDS sensor is conductivity-based.

In one embodiment, the bottom or base portion 52 of the bottle 50 is an integral portion of the bottle 50 and does not include any electronic components. In another embodiment, the bottom or base portion 52 of the bottle 50 is removable (e.g., via unscrewed a threaded connection, undoing a latch, etc.). In one embodiment, the base portion 52 of the bottle 50 contains one or more “passive” electronic components (e.g., one or more identifier tags, such as RFID or NFC, or one or more LEDs indicating a status of the water and/or a status of the cap 10). In one embodiment, the identification chip 424 is mounted under the overmolded silicone of the base.

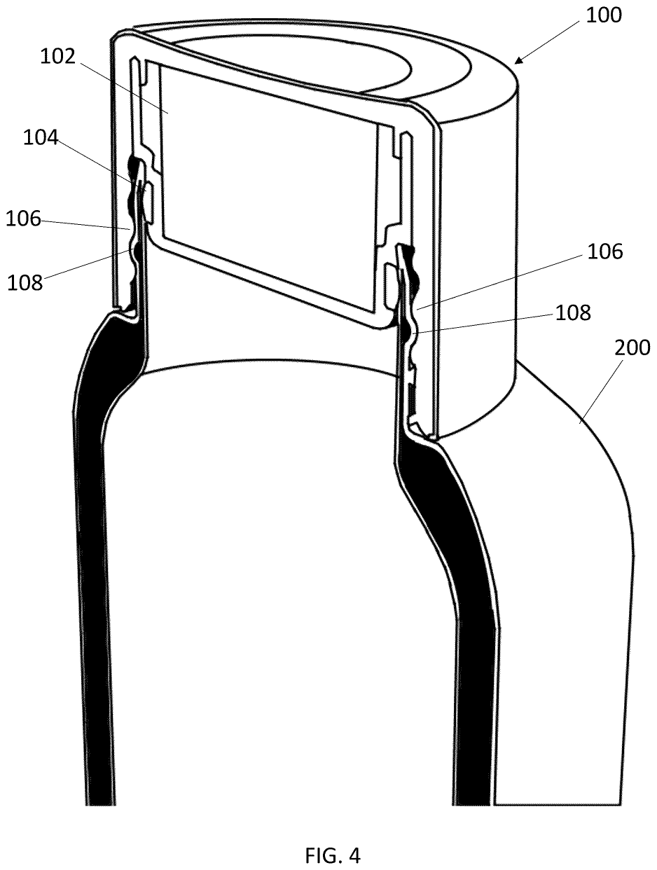

FIG. 4 illustrates a perspective sectional view of a bottle cap according to one embodiment of the present invention. In one embodiment, the bottle cap 100 includes a central stopper 102 operable to pug an opening in the top of the vessel 200. The bottle cap 100 includes cylindrical side walls extending downwardly from a top surface. In one embodiment, an inner surface of the cylindrical side walls includes a plurality of threads 106 configured to matingly engage with threads 108 positionally on the external sides of the top of the vessel 200. In one embodiment, the threads 108 are helical threads and/or annular threads. The threads allow the cap 100 to screw on the top of the vessel 200. In one embodiment, the stopper 102 includes a deformable sealing element 104 (e.g., an O-ring) surrounding an external side of a bottom portion of the stopper 102. In one embodiment, there is a small gap between the stopper 102 and the threads 106 of the internal side walls of the bottle cap 100. In one embodiment, the stopper 102 is a uniform rubber element, while, in another embodiment, the stopper includes electronic components for operating, processing, and/or powering one or more sensors. In one embodiment, one or more sensors are attached to or integrated into the bottom of the stopper 102.

In one embodiment, the one or more sensors include at last one ultrasonic transducer operable to transmit and receive ultrasonic waves in a pitch-catch orientation. The pitch-catch orientation allows distance to at least one feature (e.g., surface of a contained fluid) to be detected by time-of-flight analysis, with recognition of the fluid surface, as opposed to other features in the container being recognizable based on the impedance mismatch between the air and the fluid shown in the ultrasonic signal detected at a specific point in time. Distance between the ultrasonic transducer and the surface of the fluid allows the approximate volume of the fluid in the vessel 200 to be detected due to the known total volume of the vessel 200 and the distance to the fluid surface compared to the known height of the vessel 200 overall.

In one embodiment, the one or more sensors have an ingress protection rating of IP67. In another embodiment, the one or more sensors have an ingress protection rating of IP68. In yet another embodiment, the one or more sensors include any other ingress protection rating.

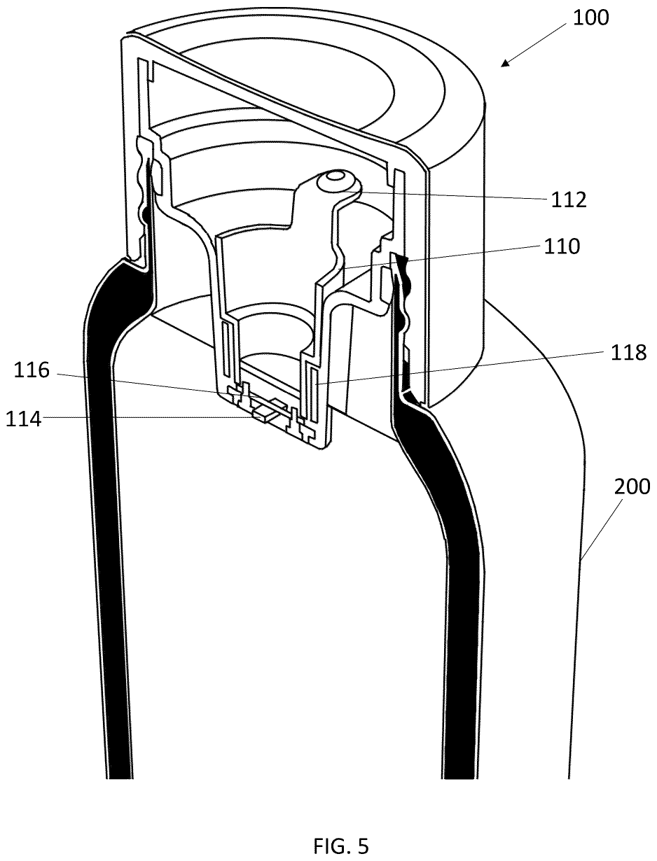

FIG. 5 illustrates a perspective sectional view of a bottle cap according to one embodiment of the present invention. In one embodiment, in addition to or in lieu of the stopper shown in FIG. 4, the bottle cap 100 includes a sensor cradle 110. In one embodiment, the sensor cradle 110 extends downwardly from a top of the bottle cap 100 and extends some length below the bottom of the bottle cap, ensuring that the bottom of the cradle 110 contacts the fluid in the vessel 200 when vessel 200 is substantially full. In one embodiment, the sensor cradle 110 is a tapered cylindrical extension. In one embodiment, the sensor cradle 110 or a portion of the sensor cradle 110 is secured to the internal side wall of the bottle cap 100 by at least one securing mechanism 112 (e.g., at least one bolt, at least one screw, by welding, etc.). In one embodiment, the bottom of the sensor cradle 110 includes at least one support 116 operable to hold at least one sensor 114. In one embodiment, the at least one sensor 114 includes at least one pH sensor, at least one TDS sensor, and/or at least one temperature sensor operable to determine one or more qualities of the fluid within. In an alternative embodiment, the at least one sensor 114 further includes a hydration sensor. In one embodiment, at least one liquid level sensor is copositioned with the at least one sensor 114 and/or on a more raised portion of the sensor cradle 110 or interior surface of the bottle cap 100. In one embodiment, the sensor cradle 110 includes at least one visual or audio indicator 118. In one embodiment, the at least one visual or audio indicator includes at least one light emitting diode operable to activate or turn a specific color when the battery of the sensor cradle 110 is low, when abnormal fluid properties (e.g., too high or low pH) are present, and/or when disconnected to an external network. In one embodiment, a portion of the vessel 200 and/or the bottle cap 100 is transparent or translucent such that the at least one light emitting diode is able to be seen when the cap is attached. In one embodiment, the at least one visual or audio indicator includes at one speaker operable to play one or more audio alerts when the battery of the sensor cradle 110 is low, when abnormal fluid properties (e.g., too high or low pH) are present, and/or when disconnected to an external network.

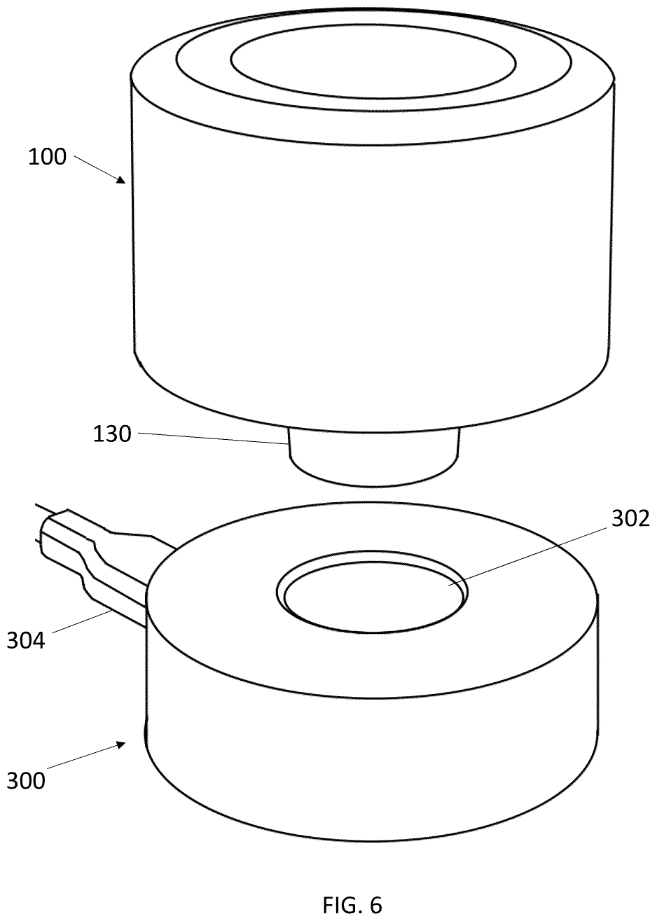

FIG. 6 illustrates a perspective view of a bottle cap and a corresponding charging system according to one embodiment of the present invention. In one embodiment, the bottle cap 100 includes an extension 130 extending downwardly from a bottom side of the bottle cap 100. In one embodiment, the extension 130 is the component that, when the bottle cap 100 is attached to the vessel, contains the sensors that detect quantities such as volume, pH, TDS, and others. In one embodiment, the extension 130 is sized and shaped to nest within a charging port 302 of a cradle 300.

In one embodiment, the cradle 300 is substantially cylindrically in shape. In one embodiment, the cradle 300 has substantially the same radius as the bottle cap 100, such that when the bottle cap 100 is nested in the cradle 300, the side walls of the cradle 300 are substantially flush with the side walls of the bottle cap 100, forming a clean, tower-like structure. In one embodiment, the cradle 300 is operable to be connected to at least one power source or at least one other external device via at least one cable 304. The at least one cable 304 is operable to be inserted into at least one power port of the cradle 300. One of ordinary skill in the art will understand that the type of port is able to be varied and includes USB-C, micro-USB, and/or any other type of cable port known in the art.

In one embodiment, the cradle 300 is operable to inductively charge the bottle cap 100 to fill the power storage used to power the components in the bottle cap 100. General methods for inductive charging are known in the art, and any such methods are compatible with the invention as recited herein. For example, in one embodiment, at least one coil is wrapped around and/or positioned beneath the charging port 302 of the cradle 300, where the at least one coil is operable to be energized with alternating current. In one embodiment, the alternating current is supplied by at least one internal power storage (e.g., a battery) within the cradle while, in another embodiment, the alternating current is supplied directly via the at least one cable 304 from at least one external power source. Other examples of inductive charging systems compatible with the present invention include, but are not limited to, those described in U.S. Pat. Nos. 11,569,685, 11,444,485, 11,398,747, 10,886,771, and 10,998,121, each of which is incorporated herein by reference in its entirety.

In one embodiment, additionally or alternatively to the use of inductive charging, the charging port 302 includes a plurality of electrical contacts positioned around the inner circumference of the charging port 302. In one embodiment, these contacts are configured to directly connect with and transmit power through one or more contacts positioned around the outer circumference of the extension 130 from the bottle cap 100. This provides a direct charging mechanism that provides for the same geometry as the inductive charging system. In one example embodiment, the charging port 302 is operable to receive wireless inductive charging power ranging from 2.6-3 watts (W). In another embodiment, the charging port 302 is operable to receive any range of power for wireless inductive charging.

In one embodiment, the cradle 300 includes at least one wireless antenna operable to communicate over a network with the bottle cap 100 and/or with at least one external server or device. In one embodiment, the cradle 300 is operable to transmit charging data to the at least one external server or device (e.g., an amount of power supplied over time, timestamp metadata for when power was supplied, magnitude of voltage drops, etc.). This data is able to be used to determine power consumption over long time periods and/or the current battery status of the bottle cap 100. In one embodiment, the cradle 300 is operable to transmit power storage data, indicating an amount of power still remaining in the cradle 300 (in embodiments where the cradle 300 includes internal power storage) to indicate when the cradle 300 needs to be connected to a power source.

In one embodiment, at least one pairing button extends outwardly from a top surface of the cradle 300 or from an external side wall of the cradle 300. In one embodiment, the at least one pairing button is flush with the surface of the cradle 300. Actuation of the at least one pairing button allows the cradle 300 to be “found” by a bottle cap 100 that is intended to be charged. In one embodiment, in order for the cradle 300 to be paired to the bottle cap 100, pairing buttons on both the cradle 300 and the bottle cap 100 need to be pressed simultaneously (or within a preset time, e.g., 1 second). In another embodiment, only buttons of the cradle 300 or the bottle cap 100 need to be pressed to pair.

In another embodiment, orientation of the bottle cap is configured such that the cradle 300 charges the bottle cap 100 from the top of the bottle cap 100, rather than the bottom. This is useful in embodiments, such as is shown in FIGS. 2A and 2B, where the battery is positioned toward the top of the cap 100 and the extension is not configured for charging. In one embodiment, a disk operable to be paired with the cradle 300 extends from a top of the bottle cap 100.

In one embodiment, the cradle 300 includes at least one LED operable to change colors to indicate a charging status of a bottle cap 100 (e.g., bottle cap is charging, bottle cap is connected but not charging, bottle cap is not connected, bottle cap is fully charged, etc.), a connection state of the cradle 300 to at least one external power source, a battery status of the cradle 300, a pairing state with at least one bottle cap 100, and/or other indications.

In one embodiment, the cradle 300 includes at least one biometric sensor. In one embodiment, the at least one biometric sensor includes at least one fingerprint scanner, at least one bioimpedance sensor, at least one retinal scanner, and/or any other suitable biometric sensor known in the art. In one embodiment, the cradle 300 only charges the bottle cap 100 if the cradle 300 detects one or more approved or known biometric signals via the at least one biometric sensor. In another embodiment, the at least one biometric sensor (e.g., the at least one bioimpedance sensor) is in the cap 100 or on a base of the bottle.

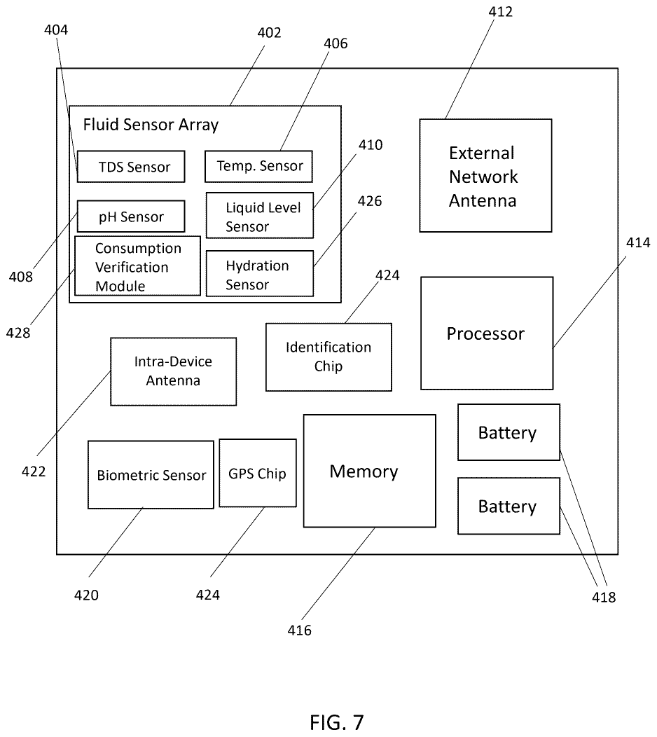

FIG. 7 illustrates a conceptual diagram of the electronic components of a bottle cap according to one embodiment of the present invention. One of ordinary skill in the art will understand that the conceptual diagram shown in FIG. 7 is not limiting as to the physical layout and orientation of the electric components of the bottle cap. The bottle cap includes a fluid sensor array 402 including one or more total dissolved solids (TDS) sensor 404, one or more temperature sensors 406, one or more pH sensors 408, one or more liquid level sensors 410, and a consumption verification module 428. In an alternative embodiment, the fluid sensor array 402 further includes one or more hydration sensors 426. The fluid sensor array 402 is configured to provide information regarding the fluid contained within the vessel and the hydration level of a user. In another embodiment, the fluid sensor array further includes a time of flight sensor.

In one embodiment, at least one external network antenna 412 is operable to transmit sensor data to at least one external server or device (e.g., smartphone, tablet, computer, smartwatch, etc.) via a network. In one embodiment, the network includes a wireless local area network (e.g., WI-FI), a wireless personal area network (e.g., BLUETOOTH), a cellular network, and/or any other type of wired or wireless network. In one embodiment the at least one external network antenna 412 is operable to receive commands to adjust one or more settings of the bottle cap and/or to receive one or more over-the-air updates to the firmware of the bottle cap.

In one embodiment, one or more sensors are positioned within a different part of the vessel (e.g., within a detachable base of the vessel) and are operable to transmit sensor data to and/or receive sensor data from the fluid sensor array 402 in the bottle cap via one or more intra-device antennas 422. In these embodiments, having sensors positioned in different locations allows sensors to be specially positioned to best provide data and prevents overcrowding of specific parts (e.g., the bottle cap), but the intra-device communication allows communication with an external device or server to go through a central point, simplifying the communications. For example, in one embodiment, the bottle cap transmits sensor data both from the fluid sensor array 402 and from other parts of the vessel (e.g., a detachable base) that is received through the intra-device antenna 422 via the external network antenna 412. In another embodiment, the external network antenna is instead positioned in another part of the device (e.g., a detachable base) that is operable to receive sensor data from the fluid sensor array 402 via the intra-device antenna 422 such that the other part of the device is the contact point with the external device or server.

In one embodiment, the cap includes a processor 414 and a memory 416. In one embodiment, the processor 414 is operable to determine basic functions, such as whether the TDS and/or pH of the liquid is above or below some minimum or maximum threshold ranges or if a hydration level is above or below some minimum or maximum threshold ranges and/or are not classified as healthy, and transmit signals to display visual or auditory warnings accordingly. The memory 416 is useful for temporarily storing sensor data, including timestamp metadata for the sensor data, before the sensor data is able to be transmitted by the external network antenna 412 to at least one external server or device (e.g., a smart phone, a tablet, a smart watch, a computer, etc.).

In one embodiment, the cap includes one or more batteries 418 operable to store charge and deliver power. In one embodiment, these batteries 418 are removable and replaceable with disposable or externally rechargeable batteries, but, in a preferred embodiment, the one or more batteries 418 act as a rechargeable power unit for the cap. One of the ordinary skill in the art will understand that the type (e.g., lithium ion, lead-acid, nickel-cadmium, lithium-sulfur, etc.), size, and/or number of batteries used in the present invention are not intended to be limiting.

In one embodiment, the cap includes at least one Global Positioning System (GPS) chip 424 or any other form of geolocation identifying sensor. The at least one GPS chip is operable to generate location data for the bottle, and is operable to correlate consumption data with the geolocation data to determine where water is consumed the most and at what locations water consumption is lacking, providing for more specific insights to the user. In one embodiment, the geolocation data is transmitted to at least one external device or server.

In one embodiment, the cap includes at least one identification chip 424, operable to be detected by at least one external sensor. In one embodiment, the at least one identification chip 424 includes at least one radiofrequency identification (RFID) chip and/or at least one near field communication (NFC) communication chip. In one embodiment, the cradle operable to charge the bottle cap includes a sensor for detecting the at least one identification chip 424. In one embodiment, the cradle and/or the cap are operable to transmit signals to at least one external device or server when the cradle detects the at least one identification chip 424, allowing for data to be produced regarding when charging begins and ends. In one embodiment, the cradle only charges the cap if the cradle detects one or more specific identification chips, allowing cradles to be specifically mated with one or more caps, while not charging others. In another embodiment, the cradle does not specifically detect identification chips and is agnostic as to the identity of the cap being charged.

In one embodiment, the cap includes at least one biometric sensor 420. By including at least one biometric sensor 420, the cap is operable to detect, based on at least one unique detected biometric identifier which user is currently drinking from the bottle. This allows multiple users to share the same bottle, or allow the main user to give small amounts of the water to someone else, while maintaining accurate data for each user. In one embodiment, the cap automatically restricts water flow through the cap if the biometric sensor 420 does not detect one or more recognized or approved users, but in another embodiment, the cap does not restrict drinking access based on the biometric sensor 420 readings. In one embodiment, the biometric sensor 420 automatically transmits an alert to at least one user device upon detection of a biometric reading that does not match one or more approved or recognized users of the bottle. In one embodiment, the biometric sensor 420 automatically transmits location data generated by the at least one GPS chip 424 if an unauthorized user is detected. In one embodiment, the at least one biometric sensor 420 includes at least one bioelectrical impedance sensor. The at least one bioelectrical impedance sensor allows, if the cap or one or more contacts extending out from the cap (e.g., adhering to an external side wall of the vessel) are contacted by a user at two points, the sensor is able to transmit a small alternating current through the body of the user and detect the returning signal to detect a unique impedance value for the user. Examples of bioimpedance sensor technology able to be included in the present invention include, but are not limited to, those described in U.S. Pat. Nos. 8,406,865 and 10,687,730 and U.S. Patent Pub. No. 2015/0157219, each of which is incorporated herein by reference in its entirety. In one embodiment, the biometric sensor 420 includes at least one fingerprint sensor, at least one retinal sensor, at least one voice detector, and/or any other form of biometric sensor known in the art.

In an alternative embodiment, the vessel and/or the bottle cap further include at least one sensor, wherein the sensor is an exterior sensor positioned on an exterior of the vessel and/or on an exterior of the bottle cap, wherein the at least one sensor is a hydration sensor, a physiological sensor, and/or any other type of sensor described herein. Throughout the present application, a hydration sensor is also referred to as a hydration sensing module. In one embodiment, the hydration sensor is an optical sensor. In one embodiment, the at least one hydration sensor includes at least one light emitting diode (LED), a light sensing module, a processor, and at least one power source. In a preferred embodiment, the at least one hydration sensor is operable to determine at least one physiological parameter upon the at least one light emitting diode contacting a user, such as, but not limited to, a finger of a user, wherein the at least one physiological parameter includes a hydration level, a heart rate (beater per minute (bpm)), a blood glucose level (BGL), and/or any other physiological measurement or parameter. Hydration sensors compatible with the present invention include, but are not limited to, those described in U.S. Patent Pub. No. 2024/0000379 and U.S. Pat. Nos. 10,966,655 and 11,690,567, each of which is incorporated herein by reference in its entirety.

In another alternative embodiment, the hydration sensor is operable to receive and/or detect contact from a user, wherein the contact includes skin contact by a user, such as a finger or hand, wherein the each of the LEDs are operable to emit pulses of light, each at a different wavelength, towards the user. In one embodiment, the light sensing module is configured to receive and/or detect the emitted light and light reflected through the skin of the user and determine wavelengths of the emitted light and the reflected light. In one embodiment, the light sensing module includes at least one detector configured to detect light which is transmitted into skin, partially absorbed by skin, and/or reflected by skin. The module is configured to generate signals corresponding to an intensity of detected light at each wavelength. In a preferred embodiment, the hydration sensor is operable to receive palm (hand palm) contact. In another embodiment, the contact includes wrist, ankle, or any other form of limb and/or skin contact. In one embodiment, the processor is operable to analyze the wavelengths of the emitted light and/or the reflected light to compute at least one physiological parameter, where the processor is further operable to compute a hydration level of the user based on the at least one physiological parameter including a hydration level, a heart rate (beats per minute (bpm)), a blood glucose level (BGL), and/or any other physiological measurement or parameter.

In one example embodiment, the smart water bottle comprises a vessel and an intelligent bottle cap, wherein the vessel is operable to contain 22 ounces (oz) of liquid, wherein the smart water bottle includes a sensor cradle comprising one or more TDS sensors 404, a temperature sensor 406, a time of flight sensor, and a consumption verification module 428. In an alternative embodiment, the smart water bottle further includes one or more pH sensors 408. In one embodiment, the smart water bottle is operable to perform intake event verification to verify when fluid intake occurs and the amount fluid ingested based on the time of flight sensor, the consumption verification module 428, a temporal correlation window, and water quality parameters (e.g., TDS, temperature, pH, or any other water quality parameters), wherein the smart water bottle must receive data from the time of flight sensor, the consumption verification module 428, the temporal correlation window, and the water quality parameters in order to perform intake event verification. In another embodiment, the smart water bottle is operable to utilize a combination of data and/or sensors in order to perform intake event verification. In one embodiment, the smart water bottle generates a verified intake event upon performing intake event verification and verifying a fluid intake occurrence.

In one embodiment, the smart water bottle vessel and/or the intelligent bottle cap includes a consumption verification module 428 comprising an accelerometer and a gyroscope operable to provide movement metrics including pitch, yaw, roll, and any other spatial movement, wherein the movement metrics indicate whether or not the bottle is resting or moving. In one embodiment, the one or more sensors momentarily stop reading upon the movement metrics indicating a moving bottle, wherein the one or more sensors resume reading upon the movement metrics indicating a resting bottle. Throughout the present application, the consumption verification module 428 is also referred to as a 6-axis (6D) inertial movement unit (IMU). In one embodiment, the consumption verification module 428 is operable to determine if the moving bottle is a sipping motion based on the movement metrics, wherein the sipping motion indicates that a user is consuming water.

In another example embodiment, the smart water bottle comprises including one or more TDS sensors 404, a temperature sensor 406, a time of flight sensor, a liquid level sensor 410, a consumption verification module 428, and further includes a wireless transceiver module operable to transmit sensor data to a server and/or a wearable device, as disclosed in further embodiments, paired with the smart water bottle. In an alternative embodiment, the smart water bottle further includes one or more pH sensors 408. In one embodiment, the server is a liquid intelligence platform, as disclosed in further embodiments. In another embodiment, the server receives a verified intake event from the smart water bottle and physiological data from the wearable device to compute an absorption factor, as disclosed in further embodiments, wherein the server is operable to determine a hydration effectiveness score (HES), as disclosed in further embodiments, by applying a convergence criterion to the absorption efficiency to utilize either publicly-derived or user-personalized absorption factor weights. In one embodiment, the user-personalized absorption factor weights are based on user metrics, as disclosed in further embodiments. In one embodiment, the HES includes a tiered confidence indicator. In one embodiment, the absorption factor is determined based on an absorption efficiency model.

In one embodiment, the smart water bottle is operable to recalibrate the one or more sensors including the TDS sensor 404, the temperature sensor 406, the consumption verification module 428, and the time of flight sensor, wherein the one or more recalibrated sensors, advantageously, are operable to provide accurate sensor data. In an alternative embodiment, the smart water bottle is further operable to recalibrate one or more pH sensors 408.

In one embodiment, the smart water bottle is operable to utilize an inference engine of the server, wherein the server is a liquid intelligence platform. In one embodiment, the smart water bottle is operable to receive public and/or private water substrate enrichment data from the liquid intelligence platform. In another embodiment, a plurality of smart water bottles are in communication with the liquid intelligence platform to perform multi-bottle networking, wherein the plurality of smart water bottles are operable to transmit data via the liquid intelligence platform.

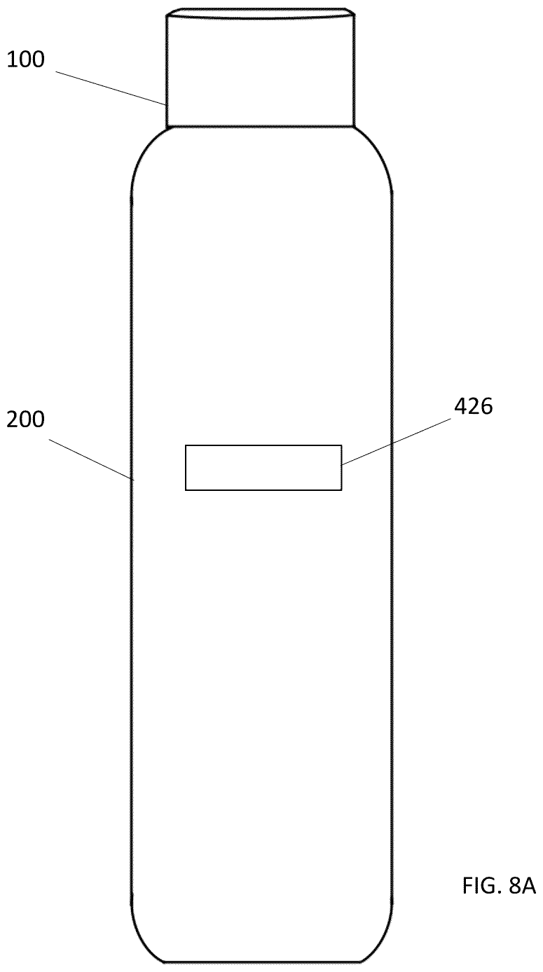

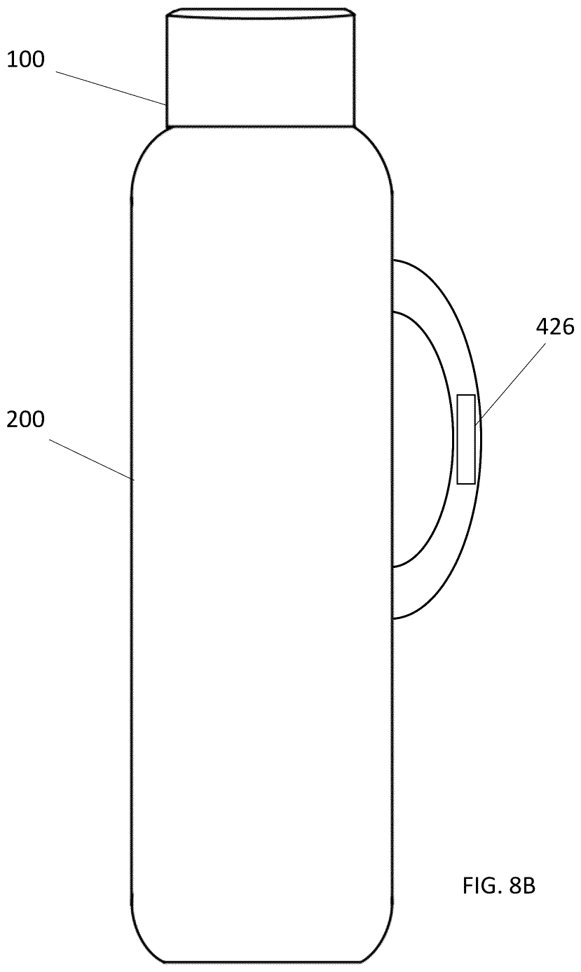

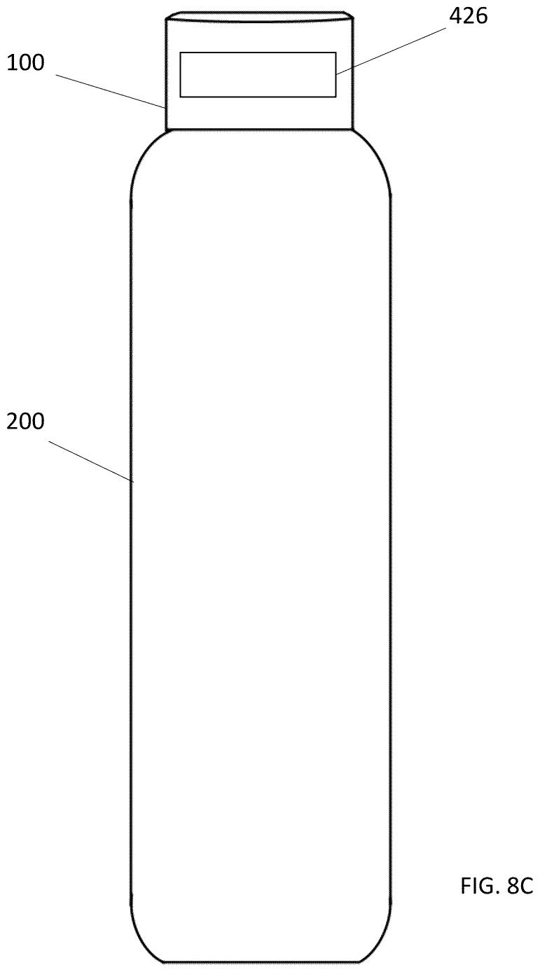

FIG. 8A illustrates a side view of a bottle including a hydration sensor according to one alternative embodiment of the present invention. In one embodiment, the hydration sensor 426 is included on a portion of an exterior side of the vessel 200. In another embodiment, the hydration sensor 426 is included on the base side of the vessel 200.

FIG. 8B illustrates a side view of a bottle including a handle and hydration sensor according to one alternative embodiment of the present invention. In one embodiment, the vessel 200 includes a handle, wherein the handle includes the hydration sensor 426.

FIG. 8C illustrates a side view of a bottle including a bottle cap with a hydration sensor according to one alternative embodiment of the present invention. In one embodiment, the hydration sensor 426 is included on an exterior side of the bottle cap. In another embodiment, the hydration sensor 426 is on the top side of the bottle cap 100. In one embodiment, the sensor cradle, as disclosed herein, is configured to retain and/or otherwise includes the hydration sensor 426.

In one embodiment, an interior of the vessel further comprises intra-device antennas operable to communicate with the bottle cap, wherein the intra-device antennas transmit the physiological parameters, and wherein the sensor cradle is operable to transmit the physiological parameters and/or hydration levels to an external device or server. In one embodiment, the intra-device communication allows communication with an external device or server to go through a central point, simplifying the communications.

In another alternative embodiment, the hydration sensor further includes at least one visual indicator and at least one audio indicator. In another embodiment, the at least one visual indicator is operable to utilize an additional LED (separate from the at least one LED) to indicate a hydration level upon the hydration sensor computing hydration level. In one embodiment, the at least one visual indicator is operable to emit a plurality of colored lights to indicate a hydration level, for example, the at least one visual indicator is operable to emit green light to indicate healthy hydration levels, emit yellow light to indicate mild dehydration, and/or emit red light to indicate significant dehydration. In one embodiment, the at least hydration sensor is operable to associate any color of the at least one visual indicator with each of the hydration levels. In one embodiment, the hydration sensor includes at least one audio indicator operable to emit an audio alert to indicate hydration level based on physiological parameters.

In an alternative embodiment, the one or more pH sensors used in the present invention include at least one glass probe sensor (e.g., an agile glass probe). However, in another embodiment, the one or more pH sensors include one or more optical sensors and/or one or more other types of pH electrode. The present invention is advantageous over the prior art, as it utilizes a sufficiently small pH sensor to be used in a bottle cap, and specifically in the extension of the bottle cap. For example, in one embodiment, the bulb of the pH sensor, such as the pH sensor shown in FIGS. 2A and 2B, is approximately 7 mm in diameter.

In a preferred embodiment, the one or more TDS sensors used in the present invention include at least one conductivity sensor. In one embodiment, the one or more TDS sensors are replaced by and/or supplemented by one or more specific analyte sensors (i.e., one or more sensors able to detect specific types of dissolved materials in the water). In one embodiment, the one or more specific analyte sensors include one or more hyperspectral sensors. In one embodiment, the one or more specific analyte sensors are able to detect the individual levels of sodium (or sodium-based salts), potassium (or potassium-based salts), calcium (or calcium-based salts), magnesium (or magnesium-based salts), and/or other types of salts or ions. In one embodiment, the one or more specific analyte sensors are able to detect the individual levels of different metals, minerals, or microplastics within the water.

In a preferred embodiment, the one or more time of flight sensors used in the present invention include at least one laser or photon-emitting time of flight sensor (e.g., formed from a single-photon avalanche diode (SPAD)). In one embodiment, the photon-emitting time of flight sensor includes a plurality of diodes (e.g., a plurality of SPADs). In another embodiment, the one or more time of flight sensors include one or more ultrasonic time of flight sensors (e.g., a piezoelectric transducer element configured to be innervated to vibrate and release ultrasonic waves and optionally able to receive ultrasonic waves reflected by the top of the water).

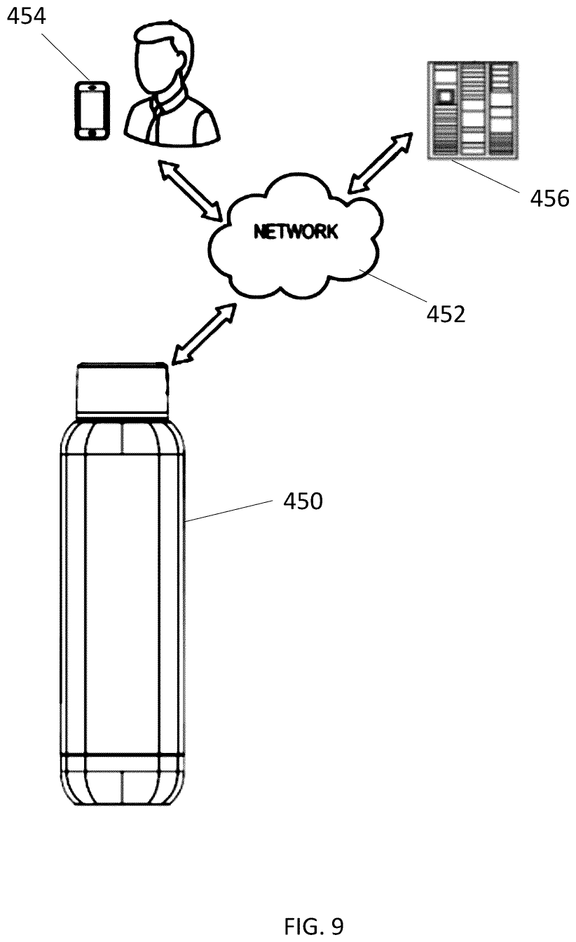

FIG. 9 illustrates a conceptual diagram of an intelligent water bottle connected to a network according to one embodiment of the present invention. In one embodiment, an intelligent water bottle 450 is connected to at least one network 452 (e.g., WI-FI network, BLUETOOTH network, cellular network, etc.). In one embodiment, at least one user device 454 is also connected to the at least one network 452 and operable to receive information concerning the intelligent water bottle 450 and/or operable to transmit commands to or about the intelligent water bottle 450. In one embodiment, the at least one network 452 includes at least one server and at least one database 456 for storing information received from the at least one user device 454 and/or the intelligent water bottle 450.

In one embodiment, data collected by the one or more pH sensors, the one or more TDS sensors, and/or the one or more temperature sensors are used to generate a water score, measuring the quality of the water within the bottle. In one embodiment, the water score is further based on data from one or more specific analyte sensors based on the presence or relative concentrations of one or more different analytes (e.g., different minerals, metals, salts, or microplastics). One of ordinary skill in the art will understand that the present invention is not intended to be limiting as to how the water score is presented. By way of example and not limitation, the water score is able to be presented as a number (out of 10, 100, etc.), a number of stars, a percentage value relative to an ideal, a series of different numbers measuring different sub-elements of the water score, or even a color on a spectrum (e.g., from red to violet). In one embodiment, a geolocation generated by a GPS chip, either on the intelligent bottle cap, or on a user device in network communication with the intelligent bottle cap, is transmitted to another user device or to one or more servers or databases. In one embodiment, the one or more servers or databases automatically correlates the geolocation with the water score. This allows the one or more servers or databases to build a state, national, global, or other geographical map of an area with associated water quality indicated for each area. In this way, the combination of the intelligent water bottle within the context of a larger network enables the development of a water quality equivalent of existing air quality maps. One of ordinary skill in the art will understand that geolocation detection is not to GPS tracking specifically, and is able to encompass other geolocating methods as well, including, but not limited to, triangulation via a plurality of WI-FI beacons, via IP geolocation, or via a cellular locator connected to the intelligent bottle cap or user device in network communication with the intelligent bottle cap, among other methods.

In one embodiment, an associated software application with the present invention is operable to transmit or generate an alert or notification on a user device when the user device enters into a geographic area with average water quality scores above or below preset thresholds.

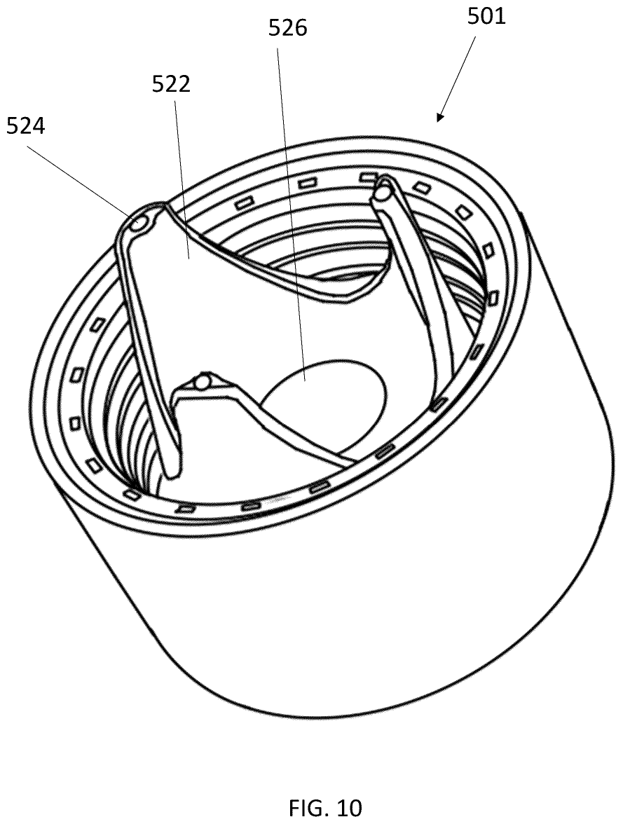

FIG. 10 illustrates a perspective view of an intelligent bottle cap 501 according to one embodiment of the present invention. In one embodiment, an intelligent bottle cap 501 according to the present invention includes a central time of flight sensor 526 surrounded by a cradle 522 having a plurality of prongs (e.g., three prongs). Additionally or alternatively, the central pH sensor 526 is able to be replaced or supplemented by one or more pH sensors and/or temperature sensors used to determine the water level within the bottle at different times. At the end of each of the prongs 522 is at least one TDS sensor 524. The prongs 522 extend far enough from the base of the cap 501 such that the at least one TDS sensor 524 dips into the water within the bottle. In this embodiment, unlike the embodiment shown in FIG. 2A, the central time of flight sensor 526 is centered in the middle of the cap 501, with the cradle 522 spanning substantially the full inner surface area of the cap 501, providing for additional space for the central sensor 526 to operate. In one embodiment, one or more of the sensors 524 at the end of the prongs 522 is a temperature sensor.

Location data is created in the present invention using one or more hardware and/or software components. By way of example and not limitation, location data is created using the Global Positioning System (GPS), low energy BLUETOOTH based systems such as beacons, wireless networks such as WIFI, Radio Frequency (RF) including RF Identification (RFID), Near Field Communication (NFC), magnetic positioning, and/or cellular triangulation. By way of example, location data is determined via an Internet Protocol (IP) address of a device connected to a wireless network. A wireless router is also operable to determine identities of devices connected to the wireless network through the router, and thus is operable to determine the locations of these devices through their presence in the connection range of the wireless router.

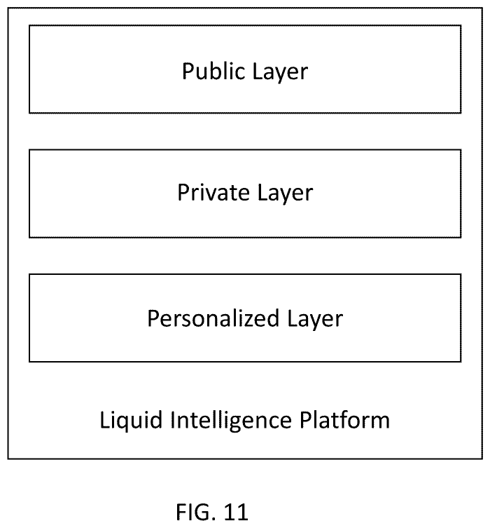

The present invention is further directed to systems and methods of integrating liquid governance, computing liquid analytics based smart devices, and calculating a net hydration score (NHS) to determine hydration levels via a liquid intelligence platform. For the purposes of this disclosure, the term liquid intelligence platform as described herein is synonymous with Water Intelligence Operating System (WIOS). FIG. 11 illustrates a diagram including three layers of the liquid intelligence platform, according to one embodiment of the present invention. In one embodiment, the liquid intelligence platform includes a personalized layer, a public layer, and a private layer.

In one embodiment, the liquid intelligence platform is operable to aggregate data from the personalized layer, the public layer, and the private layer. In another embodiment, the liquid intelligence platform is operable to provide the aggregated data from a plurality of smart devices or any other device to at least one user device and/or at least one external server. In another embodiment, the liquid intelligence platform is operable to aggregate water quality parameters, source water profiles, and/or any other data to generate reports, compute NHS, and any other function disclosed in further embodiments.

### Personalized Layer

In one embodiment, the liquid intelligence platform is operable to integrate with intelligent water bottles, wearable devices, and any other smart device, including the smart water bottle disclosed herein. In one embodiment, the liquid intelligence platform includes a hydration intelligence engine operable to compute the NHS based on holistic metrics. In another embodiment, the hydration intelligence engine calculates the NHS based on fluid intake from the intelligent water bottle, absorption factor, fluid loss, and an electrolyte adjustment index. An NHS algorithm for calculating the NHS according to one embodiment of the present invention is provided below.

$$NHS = ( \sum [ Fluid Intake ( L ) Absorption Factor ( HES ) ] ) - ( \sum [ Fluid Loss ( L ) ] ) + Electrolyte Adjustment Index ( EAI )$$

An NHS algorithm for calculating the NHS according to another embodiment of the present invention is provided below.

$$NHS = scale ( ( \sum [ VIE Volume * Absorption Factor ] ) - Hydration Demand Index ( HDI ) ) + Electrolyte Adjustment Index ( EAI )$$

In a preferred embodiment, the NHS algorithm is dependent on further embodiments related to the liquid intelligence platform and the hydration intelligence engine. In an alternative embodiment, the liquid intelligence platform utilizes the hydration sensor, as described herein, to enhance the NHS algorithm and validate the NHS. In another alternative embodiment, the liquid intelligence platform utilizes the hydration sensor, as described herein, to calculate hydration levels.

In one embodiment, the liquid intelligence platform is operable to compute fluid intake based on the amount of liquid exiting an intelligent water bottle. The intelligent water bottle includes a water bottle vessel and an intelligent water bottle cap operable to monitor bioavailability data and/or calculate fluid volume, fluid exiting the intelligent water bottle, and/or fluid quality parameters. In a preferred embodiment, the intelligent water bottle is operable to measure bioavailability of fluid based on the fluid volume, the fluid exiting the intelligent water bottle, and/or fluid quality parameters. In another embodiment, the liquid intelligence platform computes an NHS based on bioavailability. In another embodiment, the intelligent water bottle cap is operable to transform a vessel into an intelligent water bottle. In one embodiment, the intelligent water bottle cap is operable to pair with an existing water bottle vessel. In another embodiment, the vessel includes YETI, OWALA, STANLEY, CONTIGO, and HYDRO FLASK bottles.

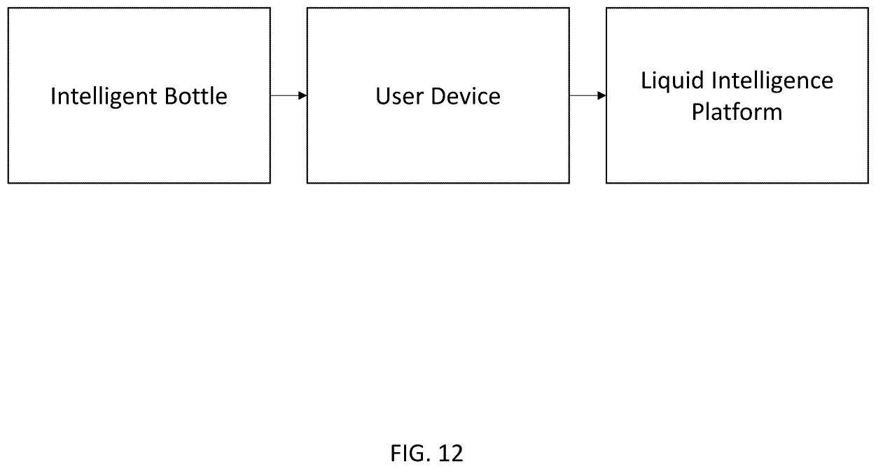

FIG. 12 illustrates a block diagram of communication between the liquid intelligence platform and the intelligent water bottle, according to one embodiment of the present invention. In one embodiment, the liquid intelligence platform is operable to receive bioavailability data from the intelligent water bottle to determine hydration levels based on the sensed data. In another embodiment, the intelligent water bottle cap is operable to compute fluid intake from the intelligent water bottle based on a difference of liquid volumes, weight, and/or any other unit of measurement before and after fluid exits the intelligent water bottle. In one embodiment, the intelligent water bottle cap is operable to compute fluid intake based on a verified intake event. In an alternative embodiment, the liquid intelligence platform is operable to receive hydration data from the intelligent water bottle based on the hydration sensor, as previously disclosed herein.

In one embodiment, the liquid intelligence platform is in communication with an intelligent water bottle, wherein the liquid intelligence platform receives bioavailability data from the intelligent water bottle, wherein the intelligent water bottle is operable to perform a bioavailability assessment to detect water quality parameters of the contained water to generate the bioavailability data, and wherein the water quality parameters include TDS, temperature, pH, dissolved oxygen (DO), and turbidity. In another embodiment, water quality parameters further include measuring the concentration of polyfluoroalkyl substances (PFAS), lead, chlorine, magnesium, calcium, sodium, chromium (e.g., Chromium-6), and other isotopes and substances within the water. In another embodiment, the liquid intelligence platform is operable to receive bioavailability data, wherein the bioavailability data includes a bioavailability index. The bioavailability index is an indicator of a physiological reaction that results from consuming a liquid with certain water quality parameters. In one embodiment, the bioavailability index determines proper hydration based on receive. In one embodiment, the liquid intelligence platform is operable to generate the bioavailability index based on the water quality parameters and data from wearable sensors, wherein the bioavailability index quantifies the usability of minerals based on the water quality parameters and data from the wearable sensors.

In another embodiment, the water quality parameters are based on the fifth unregulated contaminant monitoring rule (UCMR 5), published by the Environmental Protection Agency (EPA) on Dec. 27, 2021. However, in one embodiment, the liquid intelligence platform is operable to support any version of UCMR. In another embodiment, water quality parameters include oxidation-reduction potential (ORP).

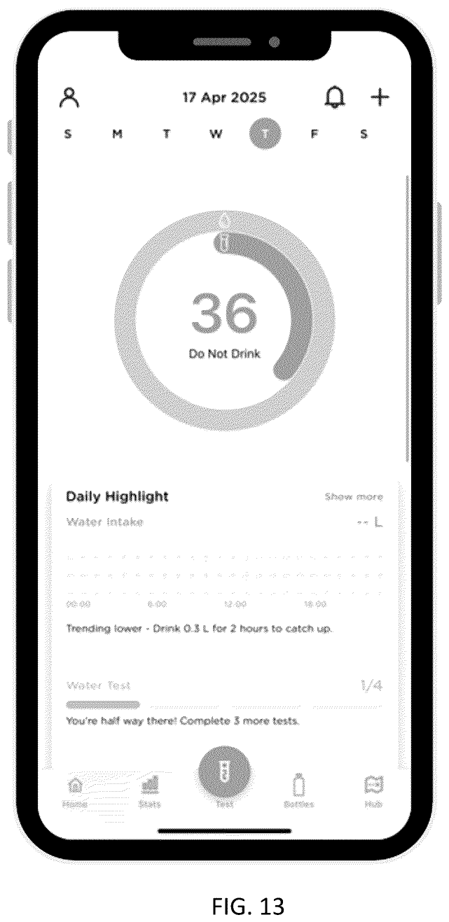

FIG. 13 illustrates a GUI for a dashboard of the liquid intelligence platform, according to one embodiment of the present invention. In one embodiment, the GUI of the liquid intelligence platform comprises a lower deck that is operable to redirect to a plurality of pages of the liquid intelligence platform, wherein the plurality of pages include, the dashboard, a statistics page, an intelligent water bottle section, and a hub section. In one embodiment, the lower deck includes the liquid test section. In one embodiment, the dashboard of the liquid intelligence platform includes an upper deck that includes a user profile, date information, and notifications. In one embodiment, the user icon includes account information.

In one embodiment, the dashboard of the liquid intelligence platform further includes a score ring, wherein the score ring displays a liquid test result and fluid intake. In another embodiment, the score ring includes an NHS displayed in the center of the score ring. In another embodiment, the score ring includes the liquid test result displayed in the center of the score ring. In one embodiment, the dashboard of the liquid intelligence platform includes a highlight section, wherein the highlight section includes a fluid intake graph and a liquid test chart. In one embodiment, the liquid intelligence platform generates the fluid intake graph, wherein the fluid intake graph illustrates fluid intake with respect to the time of day, and wherein the fluid intake graph resets daily. In another embodiment, a hydration intelligence engine of the liquid intelligence platform calculates a fluid intake trend and generates at least one recommendation based on the fluid intake trend, wherein the at least one recommendation includes an instruction to increase or decrease fluid intake. In one embodiment, the hydration intelligence engine is operable to generate the liquid test chart based on the number of liquid tests that the hydration intelligence engine performs for that day. In another embodiment, the liquid intelligence platform includes days of the week, wherein the liquid intelligence platform includes the at least one liquid test data and the NHS for each day of the week. In a preferred embodiment, the liquid intelligence platform is operable to log all contents of the dashboard to the user device and/or server based on a date, wherein the hydration intelligence engine is operable to recall the dashboard based on the date. In one embodiment, the liquid intelligence platform is operable to anonymously aggregate the contents of the dashboard for a plurality of liquid intelligence platforms to create anonymous aggregated data. In one embodiment, the liquid intelligence platform is operable to send the anonymous aggregated data to at least one health, wellness, and/or performance enterprise, wherein the health, wellness, and/or performance enterprises is granted access by the liquid intelligence platform. In another embodiment, the liquid intelligence platform is operable to send the anonymous aggregated data to any other enterprise based on a license and/or subscription.

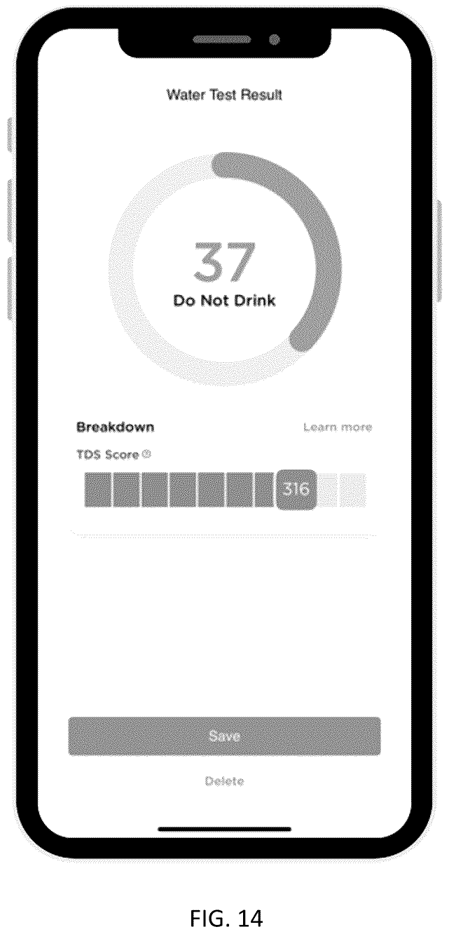

FIG. 14 illustrates a GUI for the liquid test section of the liquid intelligence platform, according to one embodiment of the present invention. In one embodiment, a hydration intelligence engine of the liquid intelligence platform receives water quality parameters from an intelligent water bottle and performs a liquid test based on the water quality parameters, wherein the liquid intelligence platform determines a liquid test result based on the liquid test. In one embodiment, the liquid test result includes a TDS score, liquid safety alert, and/or water quality parameters. In one embodiment, the liquid intelligence platform receives water quality parameters from the intelligent water bottle and generates a TDS score based on the water quality parameters. In one embodiment, the hydration intelligence engine receives the water quality parameters and lists the water quality parameters on a dropdown menu. In another embodiment, the hydration intelligence engine utilizes the TDS score to determine a liquid safety alert, wherein the liquid safety alert is operable to indicate that a liquid is safe or unsafe to drink. In one embodiment, the liquid intelligence platform receives a manual input and stores the liquid test result in the dashboard.

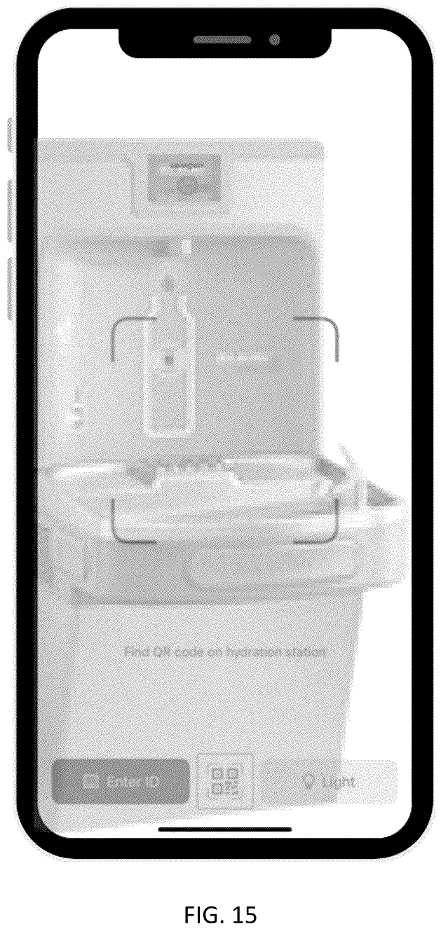

In one embodiment, the liquid intelligence platform is operable to communicate with at least one smart device via at least one hydration intelligence engine, wherein the at least one smart device is operable to detect water quality parameters, and wherein the at least one smart device is operable to create a source water profile based on the water quality parameters. FIG. 15 illustrates a GUI for a quick response (QR) scanner of the liquid intelligence platform, according to one embodiment of the present invention. In one embodiment, the liquid intelligence platform is in communication with a user device, wherein the user device includes a camera operable to perform a scan of a QR code of the at least one smart device, and wherein the liquid intelligence platform receives the scan of the QR code of the at least one smart device and initiates communication with the at least one smart device. The GUI for the QR scanner of the liquid intelligence platform comprises a guided outline to illustrate optimal QR code placement with respect to the camera of the user device for a successful scan. In one embodiment, the liquid intelligence platform provides the guided outline to the user device, wherein the liquid intelligence platform receives the scan of the QR code of the at least one smart device based on the guided outline. In another embodiment, the liquid intelligence platform receives a manual code input of the at least one smart device and initiates communication with the at least one smart device. In one embodiment, the liquid intelligence platform receives a manual input to activate the light activator, and wherein the hydration intelligence engine activates at least one light emitting source on the user device. The GUI for the QR scanner of the liquid intelligence platform includes the light activator to increase the visibility of the QR code of the at least one smart device and increase the success of the scan of the QR code of the at least one smart device.

In another embodiment, the hydration intelligence engine receives the source water profile from the at least one smart device. In one embodiment, the at least one smart device includes a hydration station, filter, fountain, sink, and/or any other water dispensing device. In another embodiment, the hydration intelligence engine receives the source water profile from a local municipality. In another embodiment, the hydration intelligence engine receives the source water profile from at least one reverse osmosis (RO) system, pump, meter, and/or valve. In yet another embodiment, the hydration intelligence engine receives the source water profile based on a manual input. In a preferred embodiment, the liquid intelligence platform aggregates the source water profile from at least one node to yield a telemetric source water profile, wherein the at least one node includes the at least one smart device, the local municipality, and/or the RO system. In another embodiment, the hydration intelligence engine receives the source water profile from an intelligent water bottle. The hydration intelligence engine is operable to utilize the source water profile and/or the telemetric source water profile to refine fluid intake from the intelligent water bottle and compute an NHS.

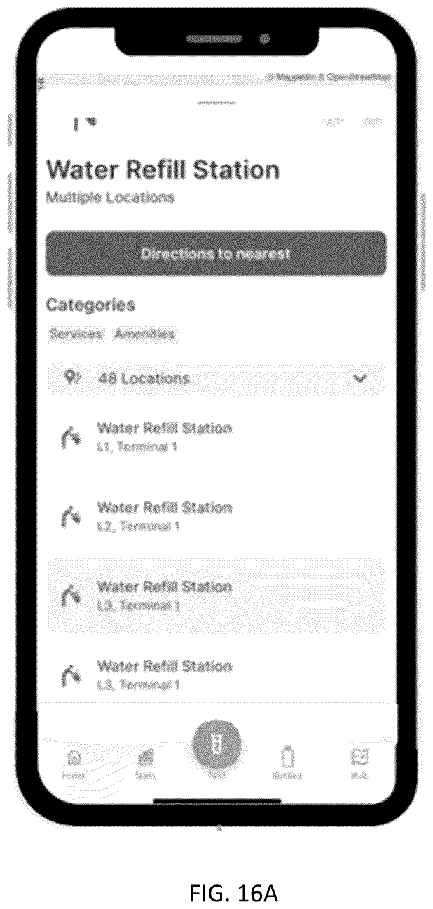

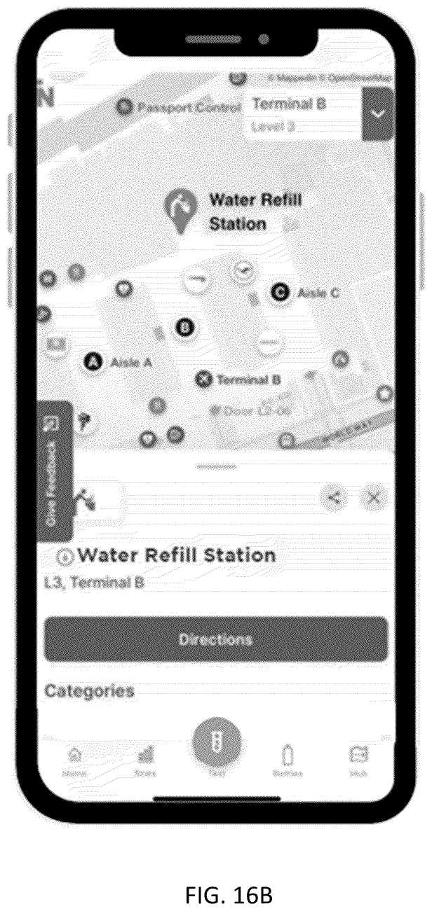

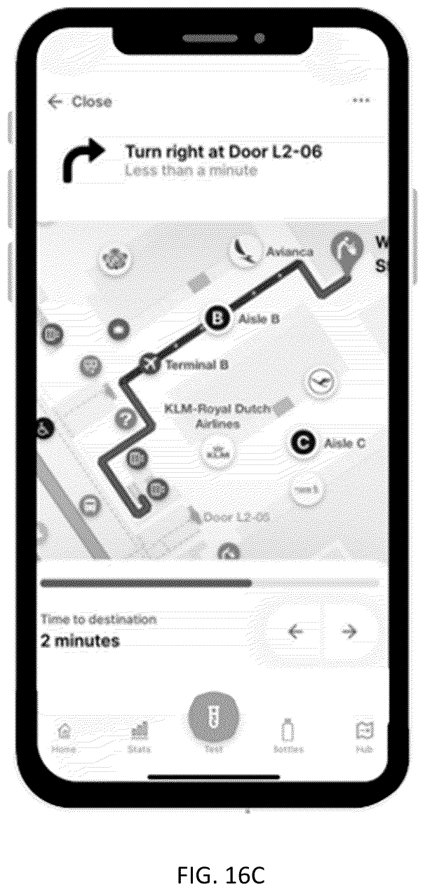

FIG. 16A illustrates a GUI for a hydration station navigation of the liquid intelligence platform, according to one embodiment of the present invention. In another embodiment, the liquid intelligence platform receives Global Positioning System (GPS) data from the intelligent water bottle and/or the user device to utilize the hydration station navigation, wherein the hydration station navigation includes at least one amenity located near the intelligent water bottle and/or the user device based on the GPS data, wherein the at least one amenity includes at least one hydration station, public water fountain, gym, cafe, etc. In one embodiment, the hydration station navigation includes a search tool to filter the at least one amenity. In another embodiment, the search tool includes categories to filter the at least one amenity. In one embodiment, the search tool includes a drop-down menu operable include at least one location for the at least one amenity.

FIG. 16B illustrates the GUI for the hydration station navigation of the liquid intelligence platform, according to one embodiment of the present invention. In one embodiment, the liquid intelligence platform receives a manual input from the user device to select the at least one amenity, wherein the hydration station navigation is operable to generate directions based on the at least one amenity selected. FIG. 16C illustrates the GUI for the hydration station navigation of the liquid intelligence platform, according to one embodiment of the present invention. In one embodiment, the liquid intelligence platform is operable to perform navigation based on the generated directions. In one example embodiment, the directions include indoor directions within a building (e.g., an airport). In another example embodiment, the directions include outdoor directions (e.g., at a university). In one embodiment, the hydration station navigation includes an expected time of arrival (ETA) based on the at least one amenity selected.

In one embodiment, the liquid intelligence platform receives an amount of liquid that at least one intelligent bottle receives from the at least one amenity to determine refill analytics. In another embodiment, the liquid intelligence platform is operable to compute an equivalent number of plastic bottles to indicate an amount of plastic saved based on the refill analytics. In one embodiment, the plastic bottle is a 16.9 fluid ounce plastic bottle.

In one embodiment, the liquid intelligence platform receives fluid intake data from a user device based on an identified food. In one embodiment, the hydration intelligence engine includes an artificial intelligence component operable to process an image and identify a food. In another embodiment, the image is a quick response (QR) code that includes nutrition facts for the identified food including electrolytes, amount of water within the identified food, etc. In one embodiment, the hydration intelligence engine receives the image and/or a manual input to determine food fluid intake, wherein the manual input includes an identification of the food (e.g., fruit, vegetable, meat, grain, tuber, etc.), quantity (weight, volume, or any other unit of food quantity measurement), preparation of the food (e.g., raw, boiled, broiled, sauteed, grilled, baked, smoked, etc.), cook temperature, and/or the amount of cook time if applicable. In one example embodiment, the hydration intelligence engine receives manual input to determine fluid intake based on 5 ounces of raw watermelon. In one embodiment, the hydration intelligence engine determines fluid intake based on a water percentage associated with each food item (e.g., watermelon (92%), chicken (65%), bread (35%), etc.).

In one embodiment, the hydration intelligence engine calculates the absorption factor based on a bioavailability index, an alkaline stability, water quality parameters, wearable sensors, food intake, and/or fluid intake, wherein the hydration intelligence engine utilizes the wearable sensors to determine a baseline based on biometric and/or physiological data, and wherein the hydration intelligence engine utilizes a frequency of the food intake and/or the fluid intake (e.g., 1cup of water every hour for the past three hours) to refine the absorption factor calculations. In one embodiment, the bioavailability index ranges from 0 to 100 or any other range of values. In another embodiment, the hydration intelligence engine calculates the absorption factor based on the presence or absence of ionic minerals (e.g., Ca, Mg, K, and/or any other mineral) and/or TDS from fluid intake and food intake. In one embodiment, the absorption factor ranges from about 0.6 to about 1.

In another embodiment, the absorption factor ranges from 0 to 1. In yet another embodiment, the absorption factor ranges from 0 to 100 or any other range of values. Because the platform provides for measurements around absorption, the present invention is operable to provide alerts and data regarding ineffective intake and recommend actions to improve intake.

In one embodiment, the hydration intelligence engine includes a bioavailability barometer operable to display an HES value in real time, wherein the HES value represents absorption efficiency. In one embodiment, the hydration intelligence engine is operable to utilize the bioavailability index, water quality parameters, source water profiles, and wearable sensors to compute the HES, wherein the bioavailability barometer is operable to display the HES value. In another embodiment, the bioavailability barometer is operable to compute and display the HES value. In one embodiment, the hydration intelligence engine is operable to calculate an absorption factor based on an HES value. An equation for the absorption factor is provided below, according to one embodiment of the present invention.

$$Absorption Factor = HES / 100$$

Fluid loss is a critical factor for the liquid intelligence platform to determine an NHS. Hydration calculations based only on fluid intake data received from an intelligent water bottle and/or received from a user device do not account for excreted fluids, which creates a false perception of hydration levels. In one embodiment, the hydration intelligence engine is operable to calculate fluid loss.

In one embodiment, the hydration intelligence engine utilizes sweat and urine metrics to calculate fluid loss. In another embodiment, the hydration intelligence engine receives data from at least one smart device to calculate fluid loss, wherein the at least one smart device is operable to provide the hydration intelligence engine sweat data and/or biometric data from wearable sensors, wherein the wearable sensors are included in APPLE, WHOOP, and OURA wearable devices. In another embodiment, the wearable sensors are operable to include wearable sensors of a tissue hydration monitor, such as those described in U.S. Patent Pub. No. 20240000379, which is incorporated herein by reference in its entirety.

In one embodiment, sweat data includes a daily, hourly, and/or per minute fluid loss based on temperature, altitude, location, and/or physical activity data. The wearable data sensor is operable to capture biometric and/or physiological data. In another embodiment, the wearable data sensor captures sweat rate, body/skin temperature, body mass index (BMI), baseline metabolic evaporation, heart rate variability (HRV) including resting heart rate and active heart rate, VO2 max, body water percentage, activity levels, sleep metrics, scale metrics, and/or other biometric and physiological metrics. In another embodiment, the hydration intelligence engine calculates fluid loss based on the at least one smart device including a smart watch, ring, phone, toilet, scale, or any other smart device. In one embodiment, the hydration intelligence engine receives fluid loss metrics from the at least one smart device including fluid loss via urine, defecation, etc. In another embodiment, the hydration intelligence engine is operable to receive fluid loss metrics via manual input from a user device. In another embodiment, the hydration intelligence engine is operable to utilize body composition sensors to validate the amount of fluid loss. In one embodiment, the liquid intelligence platform is operable to display the sweat data and/or the data from the wearable sensors in real-time. In another embodiment, the liquid intelligence platform is operable to display historical data of the data captured from the wearable sensors.

In one embodiment, the hydration intelligence engine is operable to calculate a hydration demand index (HDI) based on the biometric and/or the physiological data, wherein the HDI determines the amount of water required to hydrate a user. The HDI does not solely measure fluid loss for an individual but rather indicates how much water a user is required to drink in order to be hydrated based on activity levels, baseline health statistics, and other biometric and/or physiological metrics.

In another embodiment, the hydration intelligence engine further utilizes sauna reports to calculate fluid loss. Sauna reports include a duration and temperature of a sauna visit. In another embodiment, the liquid intelligence platform is operable to generate a body water percentage based on fluid intake and fluid loss.

In one embodiment, the liquid intelligence platform is operable to calculate an electrolyte adjustment index (EAI). In one embodiment, the hydration intelligence engine is operable to dynamically calculate an EAI based on a difference of sodium consumed and sodium lost in addition to differences of a plurality of other electrolytes including magnesium and potassium. In one embodiment, the hydration intelligence engine is operable to calculate an ideal EAI based on environmental conditions and wearable sensors, wherein the environmental conditions include temperature, humidity, altitude, weather, etc. In another embodiment, low sodium consumption with high sodium loss yields an EAI with a negative difference. In one embodiment, the hydration intelligence engine calculates an NHS based on the EAI. In an exemplary embodiment, the hydration intelligence engine calculates a higher NHS based on the ideal EAI In an exemplary embodiment, the hydration intelligence engine calculates a lower NHS based on an EAI comprising a negative difference. In one embodiment, the EAI ranges from 0 to 100 or any other range of values.

In one embodiment, the hydration intelligence engine determines a hydration level based on an NHS range, wherein the NHS is a positive or negative float value. In an alternative embodiment, the hydration intelligence engine determines a hydration level based on the NHS range, wherein the hydration intelligence utilizes hydration data and/or calculated hydration levels received from the hydration sensor of the intelligent water bottle in order to determine and/or validate the hydration level. In one embodiment, the NHS score ranges from 0 to 100 or any other range of values. In one embodiment, the NHS is greater than 1.0 which indicates an optimal hydration level. In another embodiment, the NHS ranges from about 0.3 to about 1.0 which indicates a sufficient hydration level. In another embodiment, the NHS ranges from −0.1 to 0.3 which indicates a suboptimal hydration level. In yet another embodiment, the NHS is less than −0.1 which indicates a dehydrated level. In another embodiment, the liquid intelligence platform is operable to generate an alert based on the hydration level, wherein the alert is operable to include at least one recommendation, and wherein the at least one recommendation includes instructions for increasing fluid intake, increasing mineral intake, decreasing sauna time, etc. In an exemplary embodiment, the liquid intelligence platform generates an alert indicating optimal fluid intake based on an optimal hydration level. In another exemplary embodiment, the liquid intelligence platform generates an alert that includes at least one recommendation to increase fluid intake based on the hydration level. In one embodiment, the liquid intelligence platform receives manual input to generate at least one alert periodically throughout the day.

### Public Layer

In one embodiment, the liquid intelligence platform is operable to receive governance standards from a user device and/or an external server, wherein the governance standards include EPA standards. In another embodiment, the governance standards further include data from a safe drinking water information system (SDWIS) Federal Data Warehouse, UCMR5, and enforcement and compliance history online (ECHO).

In one embodiment, the liquid intelligence platform receives a source water profile from at least one node to compute a liquid risk index, wherein the at least one node includes a municipality, and wherein the liquid intelligence platform computes the liquid risk index based on the governance standards. In one embodiment, the liquid risk index determines the probability of contamination at the at least one node. In another embodiment, the liquid risk index. In another embodiment, the liquid intelligence platform receives the source water profile to compute liquid hardness volatility, wherein the liquid hardness volatility is based on water quality parameters. In another embodiment, the liquid intelligence platform is operable to generate a report based on the source water profile and the governance standards, wherein the report includes a treatment plan, wherein the treatment plan is operable to include water supply compliance, a treatment duration, and treatment load, and wherein the treatment plan is operable to be iterative, performed in real-time, and/or dynamically adjusted based on the source water profile and the governance standards. In one embodiment, the liquid intelligence platform computes an NHS based on the treatment plan.

In one embodiment, the liquid intelligence platform receives data from at least one external source to generate the report, wherein the at least one external source includes RO systems, home water treatment control boards, and/or water leak detection systems, and wherein the report further includes benchmarks and diagnostics based on the at least one external source. The report is operable to include measured levels of contaminants, including PFAS, chromium-6, lead, bacteria, and microplastics, as well as additives such as chlorine or fluoride, and indicate levels of concern for these contaminants or additives as well as lifetime exposure levels for adults, children, pregnant women, and infants based on a number of cups of water consumed per day and a time period the person has been drinking the water. The report further includes a recommendation for a water treatment system or device, indicating the effects of using the water treatment system or device on removing contaminants or additives, such as a reduction in the levels of additives or contaminants in absolute terms (e.g., removes 2.4 ppb) or relative terms (e.g., removes 99% of a contaminant). In another embodiment, the platform is operable to provide measurements of minerals in the water, such as magnesium, calcium, and sodium, and provide recommendations for water treatment systems or devices for increasing or decreasing the minerals. Other water quality metrics are operable to be analyzed by the platform and included in the report, such as total dissolved solids (TDS), pH, or hardness. The platform is operable to provide recommendations for treatment of the water to change these metrics according to health recommendations or user preferences.

In one embodiment, the liquid intelligence platform includes an inference engine. The inference engine is able to predict at least one contamination event based on the liquid risk index, a source water profile from the at least one municipality, and/or governance standards. In one embodiment, the inference engine is operable to generate a recommendation including a treatment plan for at least one municipality to optimize water quality parameters and/or a source water profile, wherein the inference engine optimizes the water quality parameters and/or the source water profile based on governance standards. In one embodiment, the inference engine is operable to provide a third-party entity an NHS and/or body water percentage insight. In one embodiment, the liquid intelligence platform includes permissions operable to anonymize data that the inference engine provides, wherein the permissions include an opt-in permissions, anonymized aggregation of data, role-based access control (RBAC). In another embodiment, the liquid intelligence platform is operable to compute the NHS based on bioavailability data and the at least one contamination event. In one embodiment, the contamination event is based on the liquid risk index. The platform is preferably operable to integrate with third-party data sources or websites to provide real-time or near real-time alerts based on a contamination event, such as a chemical spill in a water source that acts as a supply source for a municipality. In one embodiment, the platform sends an alert in real time or near real-time to a user device (e.g., a text message or email) based on the contamination event, and includes recommendations for filtering or treating water based on the contamination event. In one embodiment, the recommendation includes a new treatment system or device. Alternatively, the recommendation includes a recommendation for servicing an existing treatment system or device. In another embodiment, the recommendation includes an action to reduce damage to a treatment system or device, or an action to source water from a different supply source until the contamination event is mitigated.

The liquid intelligence platform is also operable to retrieve data from analysis of bottled or otherwise packaged water, such as FIJI, DEER PARK, MOUNTAIN VALLEY, KIRKLAND, SMARTWATER, CRYSTAL GEYSER, and others. Regulatory history, such as FDA recalls and lawsuits are also provided by the platform. Measured contaminants, additives, minerals, water quality metrics, and other analysis, such as heat leaching from plastic (e.g., PET) are operable to be provided by the platform, with recommendations on how to treat the water to improve the water quality based on health and hydration considerations.

### Private Layer

In one embodiment, the liquid intelligence platform receives water quality parameters and/or a source water profile from a smart device, wherein the at least one smart device includes a hydration station, filter, fountain, sink, and/or any other water dispensing device. In another embodiment, the liquid intelligence platform receives water quality parameters and/or a source water profile from at least one municipality. In another embodiment, the liquid intelligence platform is operable to generate a balance index to determine if the source water profile meets optimal mineral requirements, wherein the optimal mineral requirements are determined based on governance standards. In another embodiment, the liquid intelligence platform is operable to generate an alkaline stability to determine if the source water profile meets optimal pH standards, wherein the optimal pH standards are based on the governance standards. In another embodiment, the liquid intelligence platform is operable to generate a projected depletion to determine if the filtering capacity of at least filter, wherein the filtering capacity is determined based on a decay contaminant saturation thresholds. In another embodiment, the liquid intelligence platform includes an inference engine operable to generate a report based on the balance index, alkaline stability, and/or the projected depletion, wherein the report provides a recommendation to rebalance the liquid intelligence platform, and wherein the liquid intelligence platform rebalances the balance index, the alkaline stability, and/or the projected depletion based on the recommendation. In one example embodiment, the report includes a recommendation for the liquid intelligence platform to allow liquid to enter through additional filtration stages to rebalance.

In a preferred embodiment, the liquid intelligence platform is an ecosystem including at least one node including a smart device, wherein the liquid intelligence platform provides cross-device predictive guidance based on the ecosystem.

In one embodiment, the liquid intelligence platform receives fluid intake, water quality parameters, and the source water profile from at least one node, wherein, in one embodiment, the node is an intelligent filter. In one embodiment, the liquid intelligence platform is in communication with the intelligent filter. In another embodiment, the liquid intelligence platform receives a configuration signal from the intelligent filter to pair the liquid intelligence platform with the intelligent filter.

In one embodiment, the liquid intelligence platform includes a filter dashboard, wherein the dashboard includes a filter life. In another embodiment, the liquid intelligence platform receives the filter life from the intelligent filter, wherein the filter life indicates the quality of the filter based on an amount of dispensed fluid, water quality parameters, and the source water profile. In another embodiment, the liquid intelligence platform is operable to generate at least one alert based on the filter life, wherein the alert includes a notification to dispose of and/or change the at least one intelligent filter. In another embodiment, the liquid intelligence platform is operable to receive at least one alert from the intelligent filter, wherein the alert includes a notification to dispose of and/or change the at least one intelligent filter. In another embodiment, the filter life includes mineral depletion, presence or absence of ionic minerals, and potency of ionic minerals. The platform is further operable to provide the status of a reverse osmosis membrane, carbon polish, and alkaline filter for a treatment system or device through a dashboard. In one embodiment, the liquid intelligence platform is operable to receive fluid intake and water quality parameters from the intelligent filter to calculate a hydration score.

In one embodiment, the liquid intelligence platform is operable to display filter status in real-time. In another embodiment, the liquid intelligence platform receives biometric data from a wearable sensor and the filter life to generate at least one recommendation, wherein the at least one recommendation is operable to increase an NHS. In one embodiment, the liquid intelligence platform is operable to transmit at least one alert to the intelligent filter based on the biometric data from the wearable sensor, wherein the at least one alert indicates that fluid loss increased, wherein the intelligent filter dispenses water based on the at least one alert and/or the fluid loss.

In one embodiment, the liquid intelligence platform is operable to generate a treatment plan based on water quality parameters and/or a source water profile. In one embodiment, the liquid intelligence platform transmits the treatment plan to at least one municipality, wherein the at least one treatment plan is operable to adjust the presence or the absence of ionic minerals. In one embodiment, the liquid intelligence platform is operable to calculate an absorption factor based on the treatment plan in the at least one municipality, wherein the treatment plan decreases the presence of ionic minerals in fluid in the at least one municipality. In a preferred embodiment, the liquid intelligence platform is operable to compute an NHS based on the treatment plan.

In yet another embodiment, the platform includes a service provider interface for tracking the status of treatment systems the service provider has installed, as well as opportunities for improving water quality for customers through service or upgrades as informed by water quality metrics. The dashboard integrates data from the public layer and private layer, including data from the sensors as described herein and third party sources, such as government websites and data sources (e. g, the Environmental Protection Agency). The dashboard provides alerts for treatment systems which place consumers at high risk based on time since last service and the presence of contaminants in the water supply for a geographic area that the treatment system is underequipped or not equipped to filter. Risk profiles are provided for each treatment system in one embodiment, with risks of specific contaminants and water quality issues being classified as high risk, moderate risk, or low risk. In one embodiment, risk scores are provided to quantify the risk for each consumer based on the current treatment system(s) associated with the consumer and the real-time or near real-time status of the treatment system(s). In one embodiment, the data for the platform is updated in real-time or near real-time, so that events such as a chemical spill in a river which acts as a drinking water source are accounted for by the platform in providing recommendations.

In one embodiment, the platform provides a plurality of indices or scores for a person and treatment system or device using data and measurements from the public layer, private layer, and personal layer. A municipal water risk index (MWRI) is a risk score for tap water in a specific area based on real-time or near real-time data from one or more sensors in the municipalities water system. The MWRI is operable to include insights regarding predicted trends regarding contaminants and compliance with governmental standards based on data sourced from government websites or databases. The platform is also operable to provide a softening media balance index (SMBI), which indicates if softening media inside the treatment system or device is functioning. In addition to providing the SMBI, the platform is operable to provide insights regarding the status of the media and recommendation for an action and the timing of the action to improve the SMBI. An alkaline media stability (AMS) score indicates if the system is adding the correct minerals in correct amounts. The platform provides insights relevant to mineralization and the pH of the water, indicating projected timelines relevant to service or replacement of the treatment system or device (e.g., the filter media will be depleted in 29 days and will need to be replaced). A hydration absorption efficiency (HAE) score indicates how much water a person drinks is actually being absorbed by the person. Insights provided in association with the HAE score include recommendations for treatment of water, such as a recommendation to add alkaline remineralization to raise the absorption percentage. Also referred to as a bioavailability index throughout the present application, a mineral bioavailability index (MBI), indicates if the water contains the right minerals for absorption by a consumer. The platform provides insights regarding the MBI score regarding specific minerals, including if the water includes low or high amounts of these minerals. The hydration index, discussed throughout the present application, indicates how well-hydrated the person is overall, with recommendations to improve hydration. The platform also provides insights to improve hydration, including specific actions to take to improve hydration to a specific score (e.g., 90) or threshold score (e.g., 90+).

The platform is operable to produce customized reports based on the platform data. Examples of reports include customer water quality reports, customer risk reports, maintenance schedules such as filter replacement schedules, equipment upgrade or service opportunities to treat or filter the water based on certain objectives (e.g., eliminate or reduce PFAS in water), revenue reports based on services provided, and summaries for different territories or geographic areas.

The platform of the present invention is operable to communicate with printed circuit boards (PCBs) or microchips connected to or in communication with control valves or other hardware in various locations throughout water supply systems. In one embodiment the at least one control valve is operable to include a liquid treatment sector control valve, an industrial control valve, a residential control valve, or any other liquid supplying control valve. In one embodiment, a PCB comprises at least one microchip and a memory, wherein the at least one microchip is operable to receive sensor data from at least one sensor of the control valve, wherein the at least one microchip is operable to perform edge processing and utilize artificial intelligence (AI) computing algorithms to observe and analyze water quality parameters to generate control valve data. In one embodiment, the PCB receives sensor data from the at least one sensor of the at least one control valve to analyze a usage rate of the filter as contaminants (e.g., fluoride, chloride, water softeners, or any other harmful contaminants) are filtered from the liquid exiting the control valve, wherein the usage rate ranges from 0 to 100 or any other range of values.

In one embodiment, the PCB is operable to effectively integrate with any control valve. Integrating the PCB in a plurality of control valves is advantageous because the methodology provides contamination awareness from a water supply to a drinking water source and removes blind spots for potential regions of contamination. In one embodiment, the PCB is operable to mount to the at least one control valve, wherein the PCB is in wired communication with the at least one sensor of the control valve. In another embodiment, the PCB receives sensor data from the at least one sensor of the at least one control valve via wireless communication (e.g., BLUETOOTH or any other method of wireless communication). In another embodiment, the PCB is operable to integrate with municipal water systems including sewers. In another embodiment, the PCB is operable to integrate within water systems between at least two control valves. The PCB and/or microchip provide data relating to control of water through valves, and provide for remote control of the flow of water and remote diagnostics of the control valve.

In one embodiment, the control valve data is stored locally to the memory of the PCB, wherein the control valve data is accessed from the memory of the PCB via a wired connection to at least one external computing device. In another embodiment, the control valve data is operable to be stored on a server. In another embodiment, the liquid intelligence platform is operable to utilize third-party data sources or websites to in addition to control valve data from the PCB of at least one control valve to generate a source water profile, wherein they liquid intelligence platform is operable to utilize the source water profile to generate a report to provide contamination awareness. In one embodiment, the liquid intelligence platform is operable to provide access to control valve data from the plurality of control valves based on permissions.

### AI Agent Analysis

In one embodiment, the liquid intelligence platform is operable to include at least one artificial intelligence (AI) agent. In one embodiment, the at least one AI agent includes a fluid intake AI agent, a water loss AI Agent, an absorption AI agent, an electrolyte AI agent, and a hydration AI agent. In another embodiment, the at least one AI agent includes a food moisture agent, an ionic mineral content agent, a sweat loss agent, and a heat loss agent. In another embodiment, the at least one AI agent includes a wearable data AI agent.

Utilizing AI agents enables the liquid intelligence platform to optimize the NHS calculations, alerts generated, and recommendations provided. Utilizing at least one AI agent to calculate an NHS, generate alerts, and provide recommendations without the liquid intelligence platform will fail in determining hydration levels and ensuring proper hydration. The at least one AI agent fails due to factors including a lack of an NHS calculation structure and holistic metrics for fluid intake and fluid loss provided by intelligent water bottles, smart devices, and wearable devices. Consequently, the NHS calculated, the alerts generated, and the recommendations provided by the AI Agent would result in a lack of precision and accuracy. The liquid intelligence platform receives holistic metrics and utilizes the NHS calculation structure to yield precise and accurate NHS calculations, alerts, and recommendations.

In one embodiment, the liquid intelligence platform utilizes at least one AI agent to generate the at least one alert. In another embodiment, the liquid intelligence platform is operable to provide a risk score for overhydration or dehydration based on an NHS that indicates an optimal hydration or dehydrated level. In another embodiment, the liquid intelligence platform is operable to provide the risk score based on environmental conditions, absorption factor, and data from wearable sensors. In one embodiment, the liquid intelligence platform includes a hydration streak, wherein the hydration streak tracks the number of consecutive days that the hydration intelligence engine determines an optimal and/or sufficient hydration level. In one embodiment, the at least one AI agent is operable to automatically adjust daily hydration goals based on NHS, sweat, wearable, and/or user profile metrics. In another embodiment, the at least one AI agent is operable to automatically adjust mineral needs per user based on NHS, sweat, and/or data from the wearable sensor. In another embodiment, the at least one AI agent is operable to provide at least one recommendation based on the data from the wearable sensor, wherein the recommendation includes an instruction to increase fluid intake. In yet another embodiment, the at least one AI agent is operable to automatically provide real-time feedback based on fluid intake and/or food intake. In one embodiment, the liquid intelligence platform utilizes at least one AI agent to flag electrolyte deficiency.

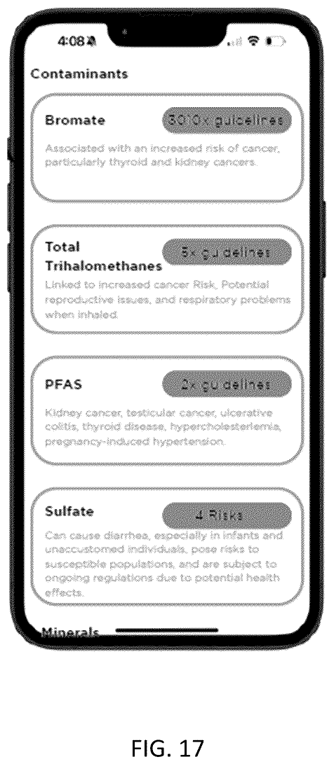

In one embodiment, the at least one AI agent is operable to source local municipality information, wherein the local municipality information includes local municipality guidelines, water quality parameters, and/or a source water profile. In one embodiment, the at least one AI agent is operable to generate at least one alert based on the local municipality information, wherein the alert indicates that the water is unsafe to drink. FIG. 17 illustrates a GUI for the contaminants page of the liquid intelligence platform, according to one embodiment of the present invention. In one embodiment, the at least one AI agent is operable to utilize a matching function to identify contaminants based on the local municipality information, wherein contaminants include bromate, trihalomethanes, PFAs, sulfates, and other isotopes and substances. In another embodiment, the at least one AI agent is operable to determine the severity of contaminants based on the local municipality information. In another embodiment, the at least one AI agent is operable to determine a future source water profile and/or the severity of contaminants based on the local municipality information.

### User Profile

In one embodiment, the liquid intelligence platform uses a system in communication with at least one server computer and at least one user device. The at least one server computer is network-based or cloud-based. The at least one server computer includes at least one processor and at least one memory. The at least one user device includes, but is not limited to, a desktop computer, a laptop computer, a smartphone, and/or a tablet. The at least one user device is operable to display a graphical user interface (GUI) for the liquid intelligence platform through a native web page, a downloaded software, and/or a mobile application. The GUI is preferably optimized for display in mobile and/or web formats. In one embodiment, the liquid intelligence platform includes a subscription based model that includes more resources for the liquid intelligence platform.

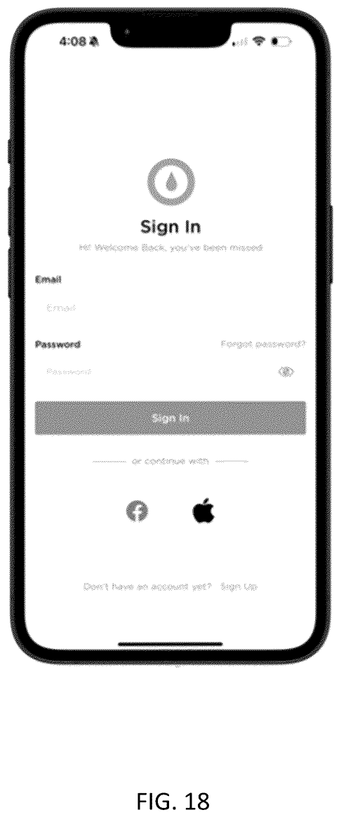

FIG. 18 illustrates a GUI for a starting page of the liquid intelligence platform, according to one embodiment of the present invention. In one embodiment, the liquid intelligence platform is operable to receive a request from at least one user device (e.g., a mobile phone, a computer, a tablet, etc.) to create an account and/or a user profile corresponding to one or more users. The liquid intelligence platform includes a plurality of accounts that are created upon registration with the liquid intelligence platform, with each account being linked to at least one user profile. Each of the at least one user profile includes account information identifying aspects of the user, user preferences, historical data for the user, and/or other data. Account information includes, but is not limited to, a name (e.g., first name, middle name, last name, nickname, username), a date of birth, a gender, an age, a race, an ethnicity, a physical address, a personal identification number (e.g., social security number (SSN), individual taxpayer identification number (ITIN)), at least one financial account (e.g., bank account number, credit card number, debit card number, digital wallet address, etc.), a job title, at least one email address, at least one phone number, a level of education, one or more alma maters, one or more additional authorized users with access to the account (e.g., a spouse, a child, etc.), electronic health records (EHRs), health information exchange (HIE) integrations, and/or other user preferences. In one embodiment, the at least one user profile includes at least one designated means of contact for recovering account information (e.g., at least one email address, at least one phone number, at least one linked social media account, etc.).

The liquid intelligence platform is dependent on user metrics received by the liquid intelligence platform from a user profile. In one embodiment, the liquid intelligence platform receives user metrics including age, gender, weight, exercise level, fluid intake including volume and source, food intake, sweat metrics, and wearable data metrics. In one embodiment, the liquid intelligence platform is operable to calculate an NHS based on the user metrics received by the liquid intelligence platform from a user profile.

### Authentication

The liquid intelligence platform is able to receive a username and a password from a user device in order to log in a user profile on the user device. In one embodiment, the liquid intelligence platform is able to receive a selection of “Forgot My Password,” which the liquid intelligence platform then generates a prompt for one or more means of communication for the liquid intelligence platform to communicate with a user device and the liquid intelligence platform receives a selection of the one or more means of communication (e.g., at least one email address, at least one phone number) from the user device. In one embodiment, the liquid intelligence platform automatically transmits a message via the one or more means of communication prompting a new password. In another embodiment, the liquid intelligence platform automatically transmits a message via the one or more means of communication including the existing username and password information to allow the user to sign in.

In one embodiment, the liquid intelligence platform receives biometric information to enable login for the user profile. Biometric information includes, but is not limited to, retinal data, a facial image scan, one or more fingerprints, and/or voice pattern recognition. In one embodiment, biometric information includes a typing test to recognize a keystroke signature previously associated with the user profile.

In one embodiment, after receiving a correct username and password, the liquid intelligence platform generates a challenge question displayed via a GUI of a user device. In one embodiment, the liquid intelligence platform prompts for answers to one or more challenge questions associated with the at least one user profile. In another embodiment, the liquid intelligence platform displayed on the GUI of the user device includes a drop down menu for selection of one or more challenge questions and subsequently receives an answer matching a question and answer associated with the user profile. In one embodiment, the drop-down menu only includes questions previously associated with the user profile. In another embodiment, the drop-down menu includes questions having answers associated with the user profile as well as questions without answers associated with the user profile, wherein the liquid intelligence platform must receive answers from the user profile for previously addressed questions. In one embodiment, rather than being used as a subsequent step to receiving a username and password, the challenge question displayed on the GUI is required to authorize the liquid intelligence platform to send an account retrieval message to a designated account recovery communication means and/or to otherwise recover a username and password.

In one embodiment, the liquid intelligence platform includes an option for signing into a user profile once the liquid intelligence platform receives confirmation that a QR code has been scanned. Examples of unique visual codes include bar codes, a quick response (QR) code, and/or any other form of visual code. After the liquid intelligence platform receives confirmation that the user device scanned the at least one unique visual code (e.g., via at least one camera and/or LiDAR sensor on the user device), the scan data is received by the liquid intelligence platform. The liquid intelligence platform verifies the validity of the at least one unique visual code and/or matches the at least one unique visual code with the user profile. If the at least one unique visual code is successfully matched, then the user profile is logged in on the scanning user device.

In one embodiment, the liquid intelligence platform includes an option to receive a log-in from at least one other user device to log on a first user device. In this embodiment, when an attempt is made to log in on the first user device, the liquid intelligence platform automatically generates and displays a unique log-in code and a prompt to visit a log-in code page on a GUI of the at least one other user device that is already signed into the user profile. The liquid intelligence platform facilitates, through the GUI of the signed in user device (e.g., a mobile phone, a smart television, a computer, tablet, a smart watch, etc.), the liquid intelligence platform receiving an input of the unique log-in code. Subsequently, the liquid intelligence platform automatically signs in the first user device to the user profile.

In one embodiment, in order to verify that the user logging in is a legitimate user and not a bot, the liquid intelligence platform generates at least one Completely Automated Public Turing test to tell Computers and Humans Apart (CAPTCHA). Upon the liquid intelligence platform receiving a selection that indicates a positive result for the at least one CAPTCHA, the user profile is successfully logged in.

In one embodiment, in order to login to a user profile, the liquid intelligence platform receives a login message from a user device, wherein the login message includes location metadata. In one embodiment, the location metadata is generated by a GPS chip found in the user device and received by the liquid intelligence platform. In one embodiment, the liquid intelligence platform authenticates or rejects the login attempt based on the associated location metadata. By way of example and not limitation, if a user from one or more restricted countries (e.g., Argentina is restricted) attempts to login to a user profile, then the liquid intelligence platform automatically rejects the login attempt. In this embodiment, the liquid intelligence platform maintains a location blacklist, designating one or more locations from which login attempts will be rejected. However, in another embodiment, the liquid intelligence platform includes a location whitelist, designating a limited number of locations from which login attempts are able to be authenticated. This is particularly useful for security purposes and ensuring, by way of example and not limitation, that only login attempts made from a user's home are able to be accepted. In one embodiment, the whitelists and/or blacklists are global whitelists and/or blacklists that apply to all user profiles on the liquid intelligence platform. In another embodiment, the whitelists and/or blacklists are specific to each user profile, meaning locations are able to be restricted or allowed with respect to specific user profiles, but not with regard to other user profiles.

In one embodiment, the login page includes at least one button for logging in via other associated social media accounts (e.g., FACEBOOK, INSTAGRAM, LINKEDIN, GMAIL, YOUTUBE, REDDIT, TWITTER, MYSPACE, TUMBLR, MASTODON, etc.). The liquid intelligence platform receives a selection of one or more social media platforms from a user device and initiates an application programming interface (API) call to verify that the user device being used to login is signed into an account on the selected one or more social media platforms on the user device. If no login using the associated social media account has previously been attempted, then the liquid intelligence platform automatically generates a user profile corresponding to the associated social media account. If an existing user profile exists corresponding to the associated social media account, then the liquid intelligence platform validates login into the existing user profile. This process is useful, as it allows a user to login and access the liquid intelligence platform without having to create a new username and password for the liquid intelligence platform.

Furthermore, in one embodiment, the liquid intelligence platform is configured to automatically retrieve data corresponding to the associated social media account via at least API call to a database of the social media platform. In one embodiment, data retrieved via these means include bibliographical data (e.g., name, address, contact information, etc.) and/or preference data (e.g., estimated political views, estimated shopping preferences, estimated content preferences, etc.). In one embodiment, retrieved bibliographical data is used to automatically fill in information corresponding to the generated user profile on the liquid intelligence platform. In one embodiment, retrieved preference data is used to filter and select media displayed for the user profile on the liquid intelligence platform.

One of ordinary skill in the art will understand that one or more of the above-mentioned authentication methods are able to be used in order for the liquid intelligence platform to log in a a user profile. In one embodiment, the liquid intelligence platform utilizes multifactor authentication, transmitting an authentication message via one or more communication means (e.g., phone number, email address, one or more linked social media accounts, etc.) including a link to authenticate a login attempt and/or a code to enter in order to complete the login attempt.

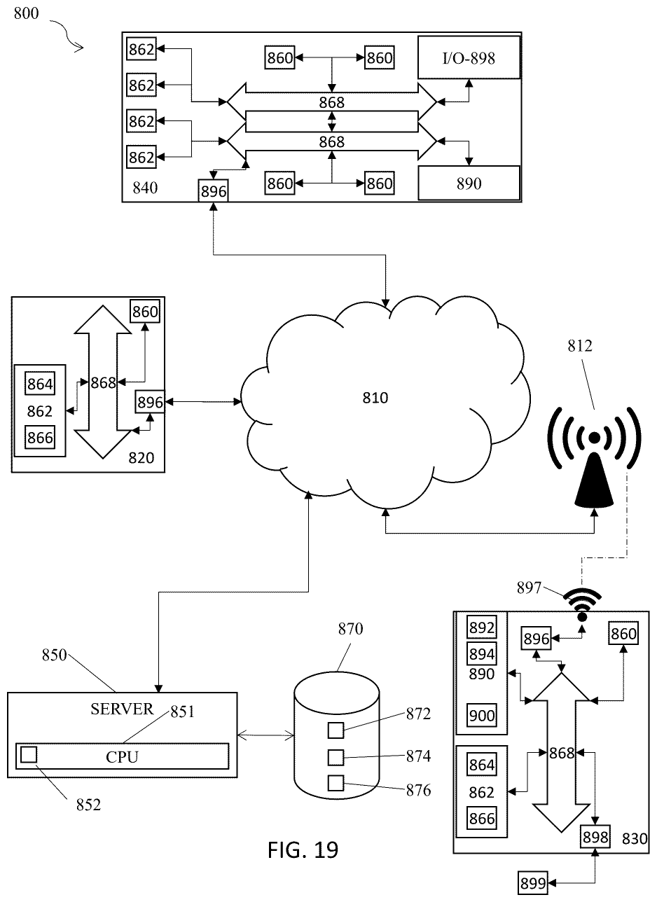

FIG. 19 is a schematic diagram of an embodiment of the invention illustrating a computer system, generally described as 800, having a network 810, a plurality of computing devices 820, 830, 840, a server 850, and a database 870.

The server 850 is constructed, configured, and coupled to enable communication over a network 810 with a plurality of computing devices 820, 830, 840. The server 850 includes a processing unit 851 with an operating system 852. The operating system 852 enables the server 850 to communicate through network 810 with the remote, distributed user devices. Database 870 is operable to house an operating system 872, memory 874, and programs 876.

In one embodiment of the invention, the system 800 includes a network 810 for distributed communication via a wireless communication antenna 812 and processing by at least one mobile communication computing device 830. Alternatively, wireless and wired communication and connectivity between devices and components described herein include wireless network communication such as WI-FI, WORLDWIDE INTEROPERABILITY FOR MICROWAVE ACCESS (WIMAX), Radio Frequency (RF) communication including RF identification (RFID), NEAR FIELD COMMUNICATION (NFC), BLUETOOTH including BLUETOOTH LOW ENERGY (BLE), ZIGBEE, Infrared (IR) communication, cellular communication, satellite communication, Universal Serial Bus (USB), Ethernet communications, communication via fiber-optic cables, coaxial cables, twisted pair cables, and/or any other type of wireless or wired communication. In another embodiment of the invention, the system 800 is a virtualized computing system capable of executing any or all aspects of software and/or application components presented herein on the computing devices 820, 830, 840. In certain aspects, the computer system 800 is operable to be implemented using hardware or a combination of software and hardware, either in a dedicated computing device, or integrated into another entity, or distributed across multiple entities or computing devices.

By way of example, and not limitation, the computing devices 820, 830, 840 are intended to represent various forms of electronic devices including at least a processor and a memory, such as a server, blade server, mainframe, mobile phone, personal digital assistant (PDA), smartphone, desktop computer, netbook computer, tablet computer, workstation, laptop, and other similar computing devices. The components shown here, their connections and relationships, and their functions, are meant to be exemplary only, and are not meant to limit implementations of the invention described and/or claimed in the present application.

In one embodiment, the computing device 820 includes components such as a processor 860, a system memory 862 having a random access memory (RAM) 864 and a read-only memory (ROM) 866, and a system bus 868 that couples the memory 862 to the processor 860. In another embodiment, the computing device 830 is operable to additionally include components such as a storage device 890 for storing the operating system 892 and one or more application programs 894, a network interface unit 896, and/or an input/output controller 898. Each of the components is operable to be coupled to each other through at least one bus 868. The input/output controller 898 is operable to receive and process input from, or provide output to, a number of other devices 899, including, but not limited to, alphanumeric input devices, mice, electronic styluses, display units, touch screens, signal generation devices (e.g., speakers), or printers.

By way of example, and not limitation, the processor 860 is operable to be a general-purpose microprocessor (e.g., a central processing unit (CPU)), a graphics processing unit (GPU), a microcontroller, a Digital Signal Processor (DSP), an Application Specific Integrated Circuit (ASIC), a Field Programmable Gate Array (FPGA), a Programmable Logic Device (PLD), a controller, a state machine, gated or transistor logic, discrete hardware components, or any other suitable entity or combinations thereof that can perform calculations, process instructions for execution, and/or other manipulations of information.

In another implementation, shown as 840 in FIG. 19, multiple processors 860 and/or multiple buses 868 are operable to be used, as appropriate, along with multiple memories 862 of multiple types (e.g., a combination of a DSP and a microprocessor, a plurality of microprocessors, one or more microprocessors in conjunction with a DSP core).

Also, multiple computing devices are operable to be connected, with each device providing portions of the necessary operations (e.g., a server bank, a group of blade servers, or a multi-processor system). Alternatively, some steps or methods are operable to be performed by circuitry that is specific to a given function.

According to various embodiments, the computer system 800 is operable to operate in a networked environment using logical connections to local and/or remote computing devices 820, 830, 840 through a network 810. A computing device 830 is operable to connect to a network 810 through a network interface unit 896 connected to a bus 868. Computing devices are operable to communicate communication media through wired networks, direct-wired connections or wirelessly, such as acoustic, RF, or infrared, through an antenna 897 in communication with the network antenna 812 and the network interface unit 896, which are operable to include digital signal processing circuitry when necessary. The network interface unit 896 is operable to provide for communications under various modes or protocols.

In one or more exemplary aspects, the instructions are operable to be implemented in hardware, software, firmware, or any combinations thereof. A computer readable medium is operable to provide volatile or non-volatile storage for one or more sets of instructions, such as operating systems, data structures, program modules, applications, or other data embodying any one or more of the methodologies or functions described herein. The computer readable medium is operable to include the memory 862, the processor 860, and/or the storage media 890 and is operable to be a single medium or multiple media (e.g., a centralized or distributed computer system) that store the one or more sets of instructions 900. Non-transitory computer readable media includes all computer readable media, with the sole exception being a transitory, propagating signal per se. The instructions 900 are further operable to be transmitted or received over the network 810 via the network interface unit 896 as communication media, which is operable to include a modulated data signal such as a carrier wave or other transport mechanism and includes any delivery media. The term “modulated data signal” means a signal that has one or more of its characteristics changed or set in a manner as to encode information in the signal.

Storage devices 890 and memory 862 include, but are not limited to, volatile and non-volatile media such as cache, RAM, ROM, EPROM, EEPROM, FLASH memory, or other solid state memory technology; discs (e.g., digital versatile discs (DVD), HD-DVD, BLU-RAY, compact disc (CD), or CD-ROM) or other optical storage; magnetic cassettes, magnetic tape, magnetic disk storage, floppy disks, or other magnetic storage devices; or any other medium that can be used to store the computer readable instructions and which can be accessed by the computer system 800.

In one embodiment, the computer system 800 is within a cloud-based network. In one embodiment, the server 850 is a designated physical server for distributed computing devices 820, 830, and 840. In one embodiment, the server 850 is a cloud-based liquid intelligence platform. In one embodiment, the cloud-based liquid intelligence platform hosts serverless functions for distributed computing devices 820, 830, and 840.

In another embodiment, the computer system 800 is within an edge computing network. The server 850 is an edge server, and the database 870 is an edge database. The edge server 850 and the edge database 870 are part of an edge computing platform. In one embodiment, the edge server 850 and the edge database 870 are designated to distributed computing devices 820, 830, and 840. In one embodiment, the edge server 850 and the edge database 870 are not designated for distributed computing devices 820, 830, and 840. The distributed computing devices 820, 830, and 840 connect to an edge server in the edge computing network based on proximity, availability, latency, bandwidth, and/or other factors.

It is also contemplated that the computer system 800 is operable to not include all of the components shown in FIG. 19, is operable to include other components that are not explicitly shown in FIG. 19, or is operable to utilize an architecture completely different than that shown in FIG. 19. The various illustrative logical blocks, modules, elements, circuits, and algorithms described in connection with the embodiments disclosed herein are operable to be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application (e.g., arranged in a different order or partitioned in a different way), but such implementation decisions should not be interpreted as causing a departure from the scope of the present invention.

Certain modifications and improvements will occur to those skilled in the art upon a reading of the foregoing description. The above-mentioned examples are provided to serve the purpose of clarifying the aspects of the invention and it will be apparent to one skilled in the art that they do not serve to limit the scope of the invention. All modifications and improvements have been deleted herein for the sake of conciseness and readability but are properly within the scope of the present invention.

The invention claimed is:

1. A water bottle, comprising:
a water bottle body including a consumption verification module;
a water bottle cap configured to attach to a top of the water bottle body;
a sensor cradle extending downwardly from a bottom surface of the water bottle cap, wherein the sensor cradle includes a plurality of prongs;
a plurality of total dissolved solids (TDS) sensors embedded in or attached to tips of the plurality of prongs; and
a time of flight sensor embedded in the sensor cradle and surrounded by the plurality of prongs;
wherein the consumption verification module comprises an accelerometer and a gyroscope, wherein the consumption verification module is configured to provide movement metrics and indicate whether or not the water bottle is resting or moving; and
wherein the water bottle is configured to perform intake event verification to verify when fluid intake occurs and the amount of fluid ingested based on the consumption verification module, the plurality of TDS sensors, and the time of flight sensor.

2. The water bottle of claim 1, wherein the sensor cradle further includes one or more temperature sensors embedded in or attached to one or more of the tips of the plurality of prongs.

3. The water bottle of claim 1, wherein the time of flight sensor is positioned approximately in a center of the sensor cradle and approximately in a center of the water bottle cap.

4. The water bottle of claim 1, wherein a top surface of the water bottle cap includes a ring light emitting diode (LED) configured to indicate battery state, connectivity state, water volume, and/or water quality.

5. The water bottle of claim 1, wherein the water bottle cap includes one or more antennas configured to transmit sensor data from the plurality of TDS sensors and/or the time of flight sensor to at least one external device and/or at least one external server.

6. The water bottle of claim 1, further including a water bottle base configured to attach to a bottom of the water bottle body, wherein the water bottle base includes an external network antenna configured to transmit sensor data to at least one external device, and wherein the water bottle base is configured to receive the sensor data from an intra-device antenna located in the water bottle cap.

7. The water bottle of claim 1, wherein the water bottle cap includes one or more batteries.

8. The water bottle of claim 1, wherein the water bottle cap includes one or more geolocation sensors.

9. A water bottle cap, comprising:
a consumption verification module comprises an accelerometer and a gyroscope, wherein the consumption verification module is configured to provide movement metrics and indicate whether or not the water bottle is resting or moving;
a sensor cradle extending downwardly from a bottom surface of the water bottle cap, wherein the sensor cradle includes a plurality of prongs;
a plurality of total dissolved solids (TDS) sensors embedded in or attached to tips of the plurality of prongs; and
a time of flight sensor embedded in the sensor cradle and surrounded by the plurality of prongs;
wherein the water bottle cap is configured to attach to a top of a water bottle body; and
wherein the water bottle is configured to perform intake event verification to verify when fluid intake occurs and the amount of fluid ingested based on the consumption verification module, the plurality of TDS sensors, and the time of flight sensor.

10. The water bottle cap of claim 9, wherein the sensor cradle further includes one or more temperature sensors embedded in or attached to one or more of the tips of the plurality of prongs.

11. The water bottle cap of claim 9, wherein the time of flight sensor is positioned approximately in a center of the sensor cradle and approximately in a center of the water bottle cap.

12. The water bottle cap of claim 9, wherein a top surface of the water bottle cap includes a ring light emitting diode (LED) configured to indicate battery state, connectivity state, water volume, and/or water quality.

13. The water bottle cap of claim 9, wherein the water bottle cap includes one or more antennas configured to transmit sensor data from the plurality of TDS sensors and/or the time of flight sensor to at least one external device and/or at least one external server.

14. The water bottle cap of claim 9, wherein the water bottle cap includes at least one bioelectrical impedance sensor configured to generate biometric data identifying a user of the water bottle cap.

15. The water bottle cap of claim 9, wherein the water bottle cap further includes one or more batteries.

16. The water bottle cap of claim 9, wherein the water bottle cap further includes one or more geolocation sensors.

17. A system including a water bottle for determining hydration effectiveness, comprising:
a water bottle body;
a water bottle cap configured to attach to a top of the water bottle body;
a water bottle base configured to attach to a bottom of the water bottle body;
one or more total dissolved solids (TDS) sensors, one or more time of flight sensors, one or more pH sensors, and/or one or more temperature sensors included within the water bottle cap;
one or more intra-device antennas positioned in the water bottle cap;
one or more external network antennas positioned in the water bottle base; and
a wearable device and a liquid intelligence platform in communication with the water bottle base;
wherein the one or more intra-device antennas are configured to transmit sensor data generated by the one or more total dissolved solids (TDS) sensors, the one or more time of flight sensors, and/or the one or more temperature sensors to the water bottle base;
wherein the water bottle includes a consumption verification module comprising an accelerometer and a gyroscope, wherein the consumption verification module is configured to provide movement metrics and indicate whether or not the water bottle is resting or moving, and wherein the movement metrics include pitch, yaw, and roll measurements of the water bottle;
wherein the one or more external network antennas are operable to receive wearable sensor data from the wearable device, wherein the wearable sensor data includes biometric and/or physiological metrics;
wherein the one or more external network antennas are configured to transmit the sensor data to the wearable device and a liquid intelligence platform, wherein the sensor data includes the wearable sensor data; and
wherein the liquid intelligence platform is operable to compute an absorption factor based on the sensor data, wherein the intelligence platform is operable to apply a convergence criterion to the absorption factor in order to compute a hydration effectiveness score (HES).

18. The water bottle of claim 17, the smart water bottle is operable to recalibrate the one or more total dissolved solids (TDS) sensors, the one or more time of flight sensors, the one or more temperature sensors, and/or the consumption verification module in order to provide accurate sensor data.

19. The water bottle of claim 17, wherein a top surface of the water bottle cap includes a ring light emitting diode (LED) configured to indicate battery state, connectivity state, water volume, and/or water quality.

20. The water bottle of claim 17, wherein the one or more sensors momentarily stop reading upon the movement metrics indicating a moving water bottle, and wherein the one or more sensors resume reading upon the movement metrics indicating a resting water bottle.
